(19) United States
(12) Patent Application Publication
(10) Pub. No.: US 2005/0250944 A1
(43) Pub. Date: Nov. 10, 2005

(54) Synthesis and uses of synephrine derivatives

(76) Inventor: Jian Chen, Diamond Bar, CA (US)

Correspondence Address: WEAN KHING WONG, 1441 HUNTINGTON DRIVE, PMB 210, SOUTH PASADENA, CA, 91030, US

(21) Appl. No.: 10/838,900

(22) Filed: May 4, 2004

(51) Int. Cl. C07D 265/30; C07D 239/70

(52) U.S. Cl. 544/170; 544/400; 548/571; 564/338

(57) ABSTRACT

The present invention discloses a novel syntheses of synephrine, its derivatives, and the salts of the foregoing, including their intermediates. One or more of the substituents of the nitrogen atom of the synephrine is/are modified to produce the derivatives. The synephrine derivatives and their salts are useful for treating animals for diseases, conditions, or disorders modulated by β₃-adrenergic receptor. They are preferably used as fat breakdown agents and/or weight loss agents.

### BACKGROUND OF THE INVENTION

Sibutramine and orlistat are synthetic drugs for treating obesity for long-term use; they were launched several years ago. Natural products such as ephedrine, pseudo-ephedrine and caffeine were once extensively used in formulations for weight-loss, but they have side effects, which include death.

In recent years, another natural product, synephrine, which is extracted from traditional Chinese medicine ZhiShi (or Citrus Aurantium), gains popularity in the market as a nutraceutical or dietary supplement for the regulation of appetite, body weight and athletic function [see e.g., Jones, U.S. Pat. No. 6,224,873B1, “Regulation of Appetite, Body Weight and Athletic Function with Materials Derived From Citrus Varieties”, issued May 1, 2001].

### SUMMARY OF THE INVENTION

One aspect of the invention presents novel syntheses of Synephrine and Synephrine Derivatives and their intermediates. The term “Synephrine Derivatives” refers to synephrine and the derivatives of synephrine represented by Formula I, below, and their salts; as well as all stereoisomers.

The synthetic pathway and Formula I are shown in Scheme 1, below, wherein “X” of Formulae III and VI is a halo (e.g., chloro, bromo, iodo and fluoro) substituent:

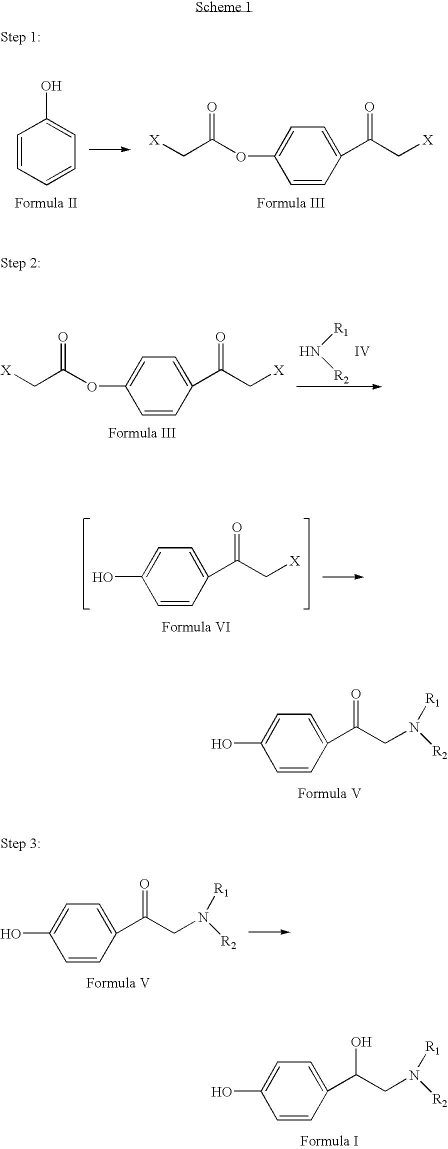

Applicant believes that the following syntheses are also new: (1) the synthesis of the compound of Formula III from that of Formula II (that is, step 1 of Scheme 1); (2) the synthesis of the compound of Formula VI from that of Formula III; and/or (3) therefore, the synthesis of the compound of Formula VI from that of Formula II. In Scheme 1, R₁ and R₂ can be the same or different. R₁ and R₂ can be separate or can be bonded together.

If R₁ and R₂ are separate, R₁ and R₂ are selected from the group consisting of:
(a) a hydrogen;
(b) a C₁ to C₆ alkyl group, the alkyl may be independently unsubstituted or substituted with 1 to 2 substituents independently selected from item (k), below;
(c) a C₁ to C₆ alkyl group which is attached to a phenyl which may be independently unsubstituted or substituted with 1 to 5 substituents independently selected from item (k), below;
(d) a C₁ to C₆ alkyl group which is attached to a 5- or 6-membered aromatic heterocyclic ring, the heterocyclic ring has 1 or 2 heteroatoms independently selected from the group consisting of O, S, and N. The 5- or 6-membered aromatic heterocyclic ring may be independently unsubstituted or substituted with 1 to 4 substituents. The substituents are independently selected from item (k), below;
(e) a C₁ to C₆ alkyl group which is attached to a 5- or 6-membered non-aromatic heterocyclic ring, the heterocyclic ring has 1 or 2 heteroatoms independently selected from the group consisting of O, S, and N. The 5- or 6-membered non-aromatic heterocyclic ring may be independently unsubstituted or substituted with 1 to 4 substituents independently selected from item (k), below;
(f) a C₃ to C₆ cycloalkyl. The cycloalkyl may be independently unsubstituted or substituted with 1 to 2 substituents independently selected from item (k), below;
(g) a C₃ to C₆ cycloalkyl group which is fused to a phenyl. The phenyl may be independently unsubstituted or substituted with 1 to 5 substituents independently selected from item (k), below;
(h) a phenyl which may be independently unsubstituted or substituted with 1 to 5 substituents independently selected from item (k), below;
(i) a 5- or 6-membered aromatic heterocyclic ring having 1 or 2 heteroatoms independently selected from the group consisting of O, S, and N. The 5- or 6-membered aromatic heterocyclic ring may be independently unsubstituted or substituted with 1 to 4 substituents independently selected from item (k), below;
(j) a 5- or 6-membered non-aromatic heterocyclic ring having 1 or 2 heteroatoms independently selected from the group consisting of O, S, and N. The 5- or 6-membered non-aromatic heterocyclic ring may be independently unsubstituted or substituted with 1 to 4 substituents independently selected from item (k), below;
(k) In items (b) to (j), above, where there are substituents, the substituents are independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine.

In the case where R₁ and R₂ are bonded together, they are bonded together to include the N of the formula (such as in Formulae I, IV, and V) to form a 5- or 6-membered heterocyclic ring. This N is counted as one heteroatom, an optional heteroatom may be included in the heterocyclic ring. The additional heteroatom is selected from the group consisting of O, S, and N. The heterocyclic ring may be independently unsubstituted or substituted with 1 to 4 substituents independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine.

The Synephrine Derivatives presented herein include both prior art and novel Synephrine Derivatives. Thus, another aspect of the present invention presents the novel Synephrine Derivatives.

Another aspect of the present invention presents the new use of both the known and new Synephrine Derivatives, as fat breakdown stimulators or body weight loss agents, preferably in animals, more preferably in mammals, and most preferably in humans.

Another aspect of the invention presents a method for treating a disease, condition or disorder modulated by β₃-adrenergic receptor in an animal, more preferably in a mammal, and most preferably in a human, that includes the step of administering to the animal or contacting the animal with a therapeutically effective amount of the known or novel Synephrine Derivatives, their salts, their solvates, or hydrates; the prodrugs of the Synephrine Derivatives, their salts, solvates or hydrates; as well as all stereoisomers. The condition includes obesity and diabetes. Also presented are pharmaceutically acceptable compositions of the foregoing.

Another aspect of the invention presents a method for screening the Synephrine Derivatives to obtain compounds which are useful as stimulators of fat breakdown or weight loss, and which preferably do not elevate blood pressure of an animal.

### BRIEF DESCRIPTION OF THE DRAWING

FIG. 1 graphically shows the effect of isopropyl-norsynephrine hydrochloride on the lipolytic activity of human adipocytes.

### DETAILED DESCRIPTION OF THE INVENTION

All publications and patent applications mentioned in this application are herein incorporated by reference to the same extent as if each of them had been individually indicated to be incorporated by reference.

### DEFINITIONS

The term “animal” includes mammal and human; mammal being the more preferred animal, and human being the most preferred animal.

The term “hydrate” refers to the complex where the solvent molecule is water.

A “pro-drug” is a compound that is transformed in vivo to yield a Synephrine Derivative, or a pharmaceutically acceptable salt, hydrate, or solvate of the Synephrine Derivative.

The term “solvate” refers to a molecular complex of a compound with one or more solvent molecules. The preferred solvent molecules are those commonly used in the pharmaceutical art, which are known to be innocuous to the animal recipient, e.g., water and ethanol.

Unless specifically identified otherwise, the term “Synephrine Derivatives” refers to Synephrine and its derivatives as represented by Formula I (shown below) and their salts; as well as all stereoisomers (including diastereoisomers and enantiomers), tautomers and isotopically lablled of the foregoing. That is, the compounds disclosed herein include both the pure stereoisomeric compounds and stereoisomeric mixtures. A non-limiting example of pure stereoisomeric compounds are optically pure isomers. Non-limiting examples of stereoisomeric mixtures are: enantiomeric mixtures and diasteromeric mixtures, in any ratio. Stereoisomeric forms are understood, in particular, as being compounds having different spatial arrangements (configurations) of the atoms or atom groups in a molecule while the atoms are linked in the same way. Thus, the Synephrine Derivatives can be in the form of pure stereoisomeric compounds or stereoisomeric mixtures. Such is also the case for the prodrugs of the Synephrine Derivatives; the salts, solvates and hydrates of the Synephrine Derivatives and their prodrugs. R₁ and R₂ are defined further below.

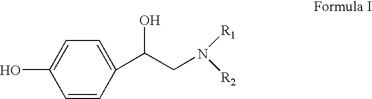

“Bamethan” is the common name for the chemical name “2-butylamino-1-(4-hydroxyphenyl)-ethanol” represented by Formula (7), below:

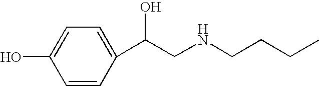

### Bamethan, or 2butylamino-1-(4-hydroxyphenyl)-ethanol Formula (7)

“Ethylnorsynephrine” is the common name for the chemical name “2-ethylamino-1-(4-hydroxyphenyl)-ethanol” represented by Formula (5), below:

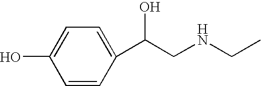

### Ethylnorsynephrine, or 2-ethylamino-1-(4-hydroxyphenyl)-ethanol Formula (5)

“Isopropylnorsynephrine” and “N-isopropyloctopamine” are the common names for the chemical name “2-isopropylamino-1-(4-hydroxyphenyl)-ethanol” represented by Formula (1), below:

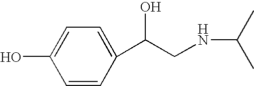

### Isopropylnorsynephrine, N-isopropyloctopamine, or 2-isopropylamino-1-(4-hydroxyphenyl)-ethanol Formula (1)

“Octopamine”, also known as “norsynephrine”, is the common name for the chemical name “2-amino-1-(4-hydroxyphenyl)-ethanol” represented by Formula (6), below:

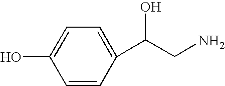

### Octopamine, norsynephrine, or 2-amino-1-(4-hydroxyphenyl)-ethanol Formula (6)

“phenyl-t-butylnorsynephrine” is the common name for the chemical name “2-(phenyl-t-butylamino)-1-(4-hydroxyphenyl)-ethanol” represented by Formula (9), below:

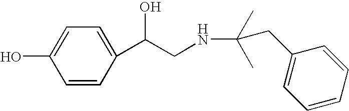

### Phenyl-t-butylnorsynephrine, or 2-(phenyl-t-butylamino)-1-(4hydroxyphenyl)-ethanol Formula (9)

“Propylnorsynephrine” is the common name for the chemical name “2-propylamino-1-(4-hydroxyphenyl)-ethanol” represented by Formula (8), below:

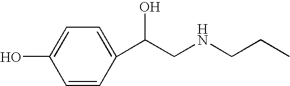

### Propylnorsynephrine, or 2-propylamino-1-(4-hydroxyphenyl)-ethanol Formula (8)

“tert-Butylnorsynephrine” is the common name for the chemical name “2-tert-butylamino-1-(4-hydroxyphenyl)-ethanol” represented by Formula (3), below:

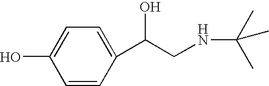

### tert-Butylnorsynephrine, or 2-tert-butylamino-1-(4-hydroxyphenyl)-ethanol Formula (3)

“Synephrine” is the common name for the chemical name “2-methylamino-1-(4-hydroxyphenyl)-ethanol” represented by Formula (4), below:

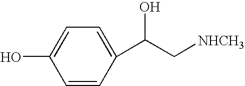

### Synephrine, or 2-methylamino-1-(4-hydroxyphenyl)-ethanol Formula (4)

The term “agonist” refers to both full and partial agonists.

The phrase “modulated by a β₃-adrenergic receptor” or “modulation of a β₃-adrenergic receptor” refers to the activation or deactivation of β₃-adrenergic receptors. For example, a compound which modulates a β₃-adrenergic receptor may act as an agonist, partial agonist, inverse agonist, antagonist, partial antagonist, and the like.

The phrase “pharmaceutically acceptable” indicates that the substance or composition is compatible chemically and/or toxicologically with the other ingredients comprising a formulation and/or the animal being treated therewith.

The term “treating”, “treat”, or “treatment” embrace both preventative, i.e., prophylactic, and palliactive treatment.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

Synephrine Derivatives and Methods for Synthesizing Them

Some of the known derivatives of synephrine (such as the naturally occurring octopamine) can be obtained from natural quantities since they occur in very low amount in nature. Some of them can be obtained through semi-synthesis starting from the naturally occurring octopamine, and some are obtained from organic synthesis.

Adamski et al. [United States patent No. (hereinafter referred to as “U.S. Pat. No.”) 3,860,651, “Reductive Alkylation of Amines”, issued Jan. 14, 1975] states in its Abstract that “An improved process for the production of paraproterenol and related compounds is achieved by reacting a ketone with the hydrochloride salt of an alkanol amine in the presence of hydrogen and a palladium on charcoal catalyst.” Paraproterenol is also known as isopropylnorsynephrine or isopropyloctopamine. The method of Adamksi et al., is actually a semi-synthesis of isopropylnorsynephrine from octopamine, but the natural source of octopamine is very rare. Thus, a method for making octopamine itself, especially an efficient method, is needed.

Moore et al [Moore et al, U.S. Pat. No. 2,460,144, “Hydroxyphenyl Alkanolamines”, issued Jan. 25, 1949] describes the preparation of hydroxyphenyl alkanolamines by the reduction of (hydroxyphenyl)-aminoalkyl ketone with palladium catalyst and hydrogen in water solvent. However, Moore et al does not mention how to obtain the (hydroxyphenyl)-aminoalkyl ketone. The method disclosed in Moore et al is limited. Catalytic hydrogenation method cannot be used for substance possessing sensitive substituted group on the nitrogen atom, such as the benzyl group.

Fodor et al., [G. Fodor, et al., “A synthesis of adrenaline-like compounds”, J. Am. Chem. Soc., 71:1045-8 (1949)] describes the synthesis of adrenaline-like compounds by oxidation of p-HOC₆H₄COCH₃ to p-OHC₆H₄COCHO with SeO₂, followed by reductive alkylation with amine. In this method, p-HOC₆H₄Ac is not a very basic chemical, and SeO₂ is expensive.

Asscher et al., reports a Houben-Hoesch reaction of phenol or its derivatives with aminoacetonitriles, with the help of gaseous HCl and ZnCl₂ or AlCl₃ to synthesize α-amino-p-hydroxyacetophenones, followed by a catalytic reduction of the ketone to the corresponding aminoethanols [M. Asscher et al., “A new synthesis of α-amino-p-hydroxyacetophenones and their reduction to the corresponding aminoethanols”, Neth. Rec. Trav. Chim, 68: 960-8 (1949)]. Asscher et al.'s synthetic method appears to be facile; however, the extremely toxic chemical NaCN has to be used, toxic solvents such as PhNO₂ and PhCl are also used, and the preparation of aminoacetonitriles is not easy. Two patents reported a similar method: Asscher et al., U.S. Pat. No. 2,585,988, “Method of Preparing Aminoketones”, issued Feb. 19, 1952; and Grayson et al., European Patent Appl. No. 0 431 871 A2, “A process for the preparation of ketones”, publ. Jun. 12, 1991.

Molteni et al., describes yet another synthesis which uses hydroxybenzoic acids or their chlorides as starting materials [Molteni et al., GB Patent Specification No.1,425,049, “Substituted 2-Hydroxy-2-Phenylethylamines”, specification publ. Feb. 18, 1976]. This synthesis requires five or six steps to obtain the substituted 2-aminoalkanol. Further, the preparation of diazomethane is not easy to handle.

Satzinger at al. reports the preparation of intermediate arylisonitrosoalkanone from alkyl nitrite and p-hydroxyacetophenone, followed by their reduction to substituted 2-aminoalkanol (such as octopamine). However, the reduction reaction had to be carried out under strong acidic condition, which was not good to the equipments, and which used expensive solvents (DMF, HMPT, DMSO) [Satzinger et al., U.S. Pat. No. 3,966,813, “Process for the Preparation of 1-(m- AND P-Hydroxyphenyl)-2-Aminoethanol”, issued Jun. 29, 1976]. Another patent reports a similar method: Tafesh et al., U.S. Pat. No. 5,466,873, “Process for Preparing Arylketoamines”, issued Nov. 14, 1995.

Thus, it is clear from the above that synephrine, octopamine and their derivatives are difficult to manufacture and there does not exist a common method nor a practicable method for the production of these compounds in industry.

Thus, one aspect of the present invention presents a novel, relatively easy and practicable synthesis (as shown in Scheme 1) of the derivatives of synephrine which includes synephrine and compounds similar to synephrine except for the modification on the nitrogen atom of synephrine. Unless specifically identified otherwise, the term “Synephrine Derivatives” refers to synephrine and its derivatives as represented by Formula I (shown below), and their salts; as well as all stereoisomers (including diastereoisomers and enantiomers), tautomers and isotopically lablled of the foregoing. That is, the compounds disclosed herein include both the pure stereoisomeric compounds and stereoisomeric mixtures. A non-limiting example of pure stereoisomeric compounds are optically pure isomers. Non-limiting examples of stereoisomeric mixtures are: enantiomeric mixtures and diasteromeric mixtures, in any ratio. Stereoisomeric forms are understood, in particular, as being compounds having different spatial arrangements (configurations) of the atoms or atom groups in a molecule while the atoms are linked in the same way. Thus, the Synephrine Derivatives can be in the form of pure stereoisomeric compounds or stereoisomeric mixtures. Such is also the case for the prodrugs of the Synephrine Derivatives; the salts, solvates and hydrates of the Synephrine Derivatives and their prodrugs.

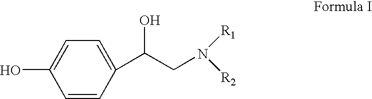

Thus, even though for ease of discussion, in the following discussion only Formula I is used, it is understood that the salts thereof are also being claimed. Additionally, even though the examples of the Synephrine Derivatives are usually presented in the forms of their free-base formulae, it is to be understood that the salt forms of the Synephrine Derivatives are also included in the definition of “Synephrine Derivatives”. Thus, in the discussion of the compounds, their uses, and synthesis, it is understood that the salt and free base forms of the Synephrine Derivatives may be used. For example, the pharmaceutically acceptable salts of the Synephrine Derivatives are presented, non-limiting examples of which are non-toxic salts of the Synephrine Derivatives, such salts include those derived from organic and inorganic acids such as, without limitation, hydrochloric acid, hydrobromic acid, phosphoric acid, sulfuric acid, methanesulfonic acid, acetic acid, dichloroacetic acid, tartaric acid, lactic acid, succinic acid, citric acid, maleic acid, fumaric acid, sorbic acid, aconitic acid, salicylic acid, and phthalic acid. These salts can be prepared in situ during the final isolation and purification of the Synephrine Derivatives, or by separately reacting a Synephrine Derivative with a suitable organic or inorganic acid (such as those listed in this paragraph) and isolating the salt thus formed; the foregoing can be achieved, e.g., by using methods known in the art.

In the following description of R₁ and R₂ of Formula I, some chemical structures are used to provide non-limiting examples of the possible chemical structures of the Synephrine Derivatives.

In Formula I, R₁ and R₂ can be the same or different. Also, R₁ and R₂ can be separate or can be bonded together.

If R₁ and R₂ are separate, R₁ and R₂ are independently selected from the group consisting of:
(a) a hydrogen;
E.g., the Synephrine Derivative represented by the formula below: 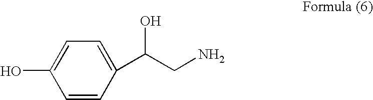
(b) a C₁ to C₆ alkyl group, the alkyl may be independently unsubstituted or substituted with 1 to 2 substituents independently selected from item (k), below;
E.g., the Synephrine Derivatives represented by the formulae below: 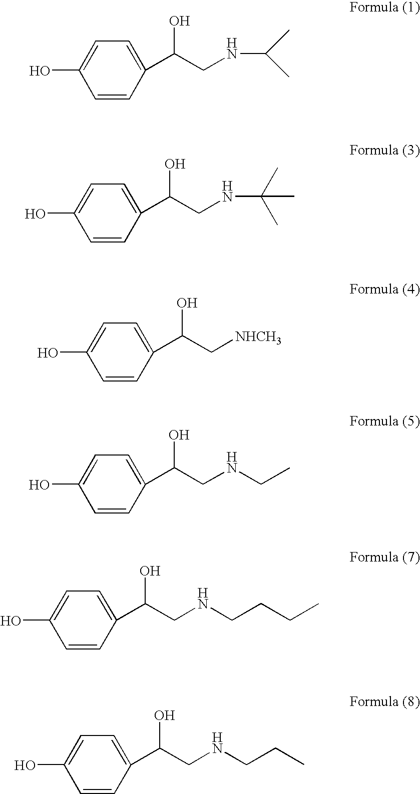
(c) a C₁ to C₆ alkyl group which is attached to a phenyl which may be independently unsubstituted or substituted with 1 to 5 substituents independently selected from item (k), below;
E.g., the Synephrine Derivatives represented by the formulae below: 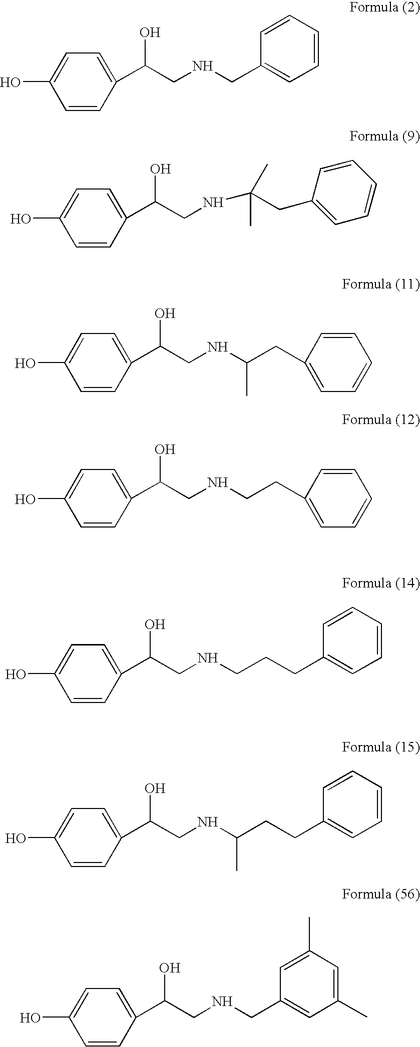
(d) a C₁ to C₆ alkyl group which is attached to a 5- or 6-membered aromatic heterocyclic ring, the heterocyclic ring has 1 or 2 heteroatoms independently selected from the group consisting of O, S, and N. The 5- or 6-membered aromatic heterocyclic ring may be independently unsubstituted or substituted with 1 to 4 substituents. The substituents are independently selected from item (k), below;
E.g., the Synephrine Derivatives represented by the formulae below: 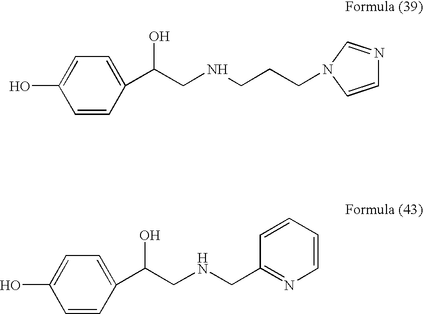
(e) a C₁ to C₆ alkyl group which is attached to a 5- or 6-membered non-aromatic heterocyclic ring, the heterocyclic ring has 1 or 2 heteroatoms independently selected from the group consisting of O, S, and N. The 5- or 6-membered non-aromatic heterocyclic ring may be independently unsubstituted or substituted with 1 to 4 substituents independently selected from item (k), below;
E.g., the Synephrine Derivatives represented by the formulae below: 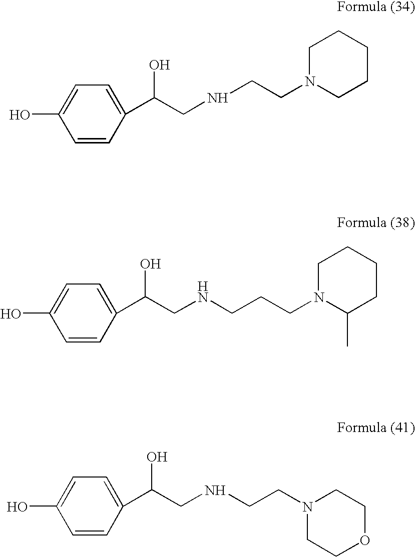
(f) a C₃ to C₆ cycloalkyl. The cycloalkyl may be independently unsubstituted or substituted with 1 to 2 substituents independently selected from item (k), below;
E.g., the Synephrine Derivatives represented by the formulae below: 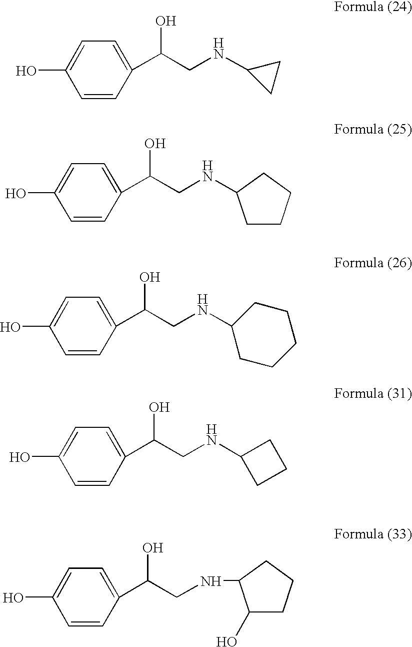
(g) a C₃ to C₆ cycloalkyl group which is fused to a phenyl. The phenyl may be independently unsubstituted or substituted with 1 to 5 substituents independently selected from item (k), below;
E.g., the Synephrine Derivative represented by the formula below: 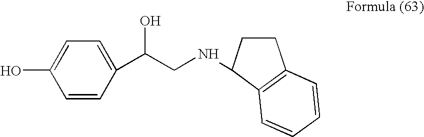
(h) a phenyl which may be independently unsubstituted or substituted with 1 to 5 substituents independently selected from item (k), below;
E.g., the Synephrine Derivatives represented by the formulae below: 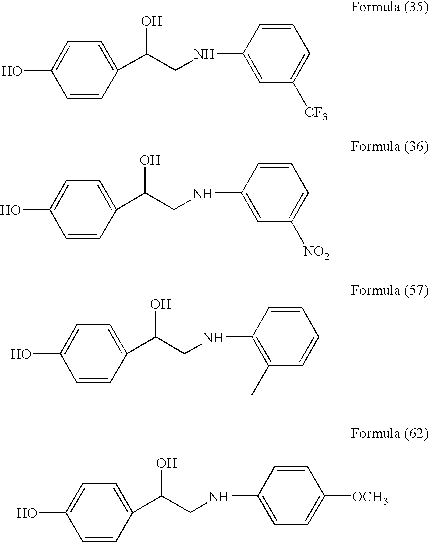
(i) a 5- or 6-membered aromatic heterocyclic ring having 1 or 2 heteroatoms independently selected from the group consisting of O, S, and N. The 5- or 6-membered aromatic heterocyclic ring may be independently unsubstituted or substituted with 1 to 4 substituents independently selected from item (k), below;
E.g., the Synephrine Derivatives represented by the formulae below: 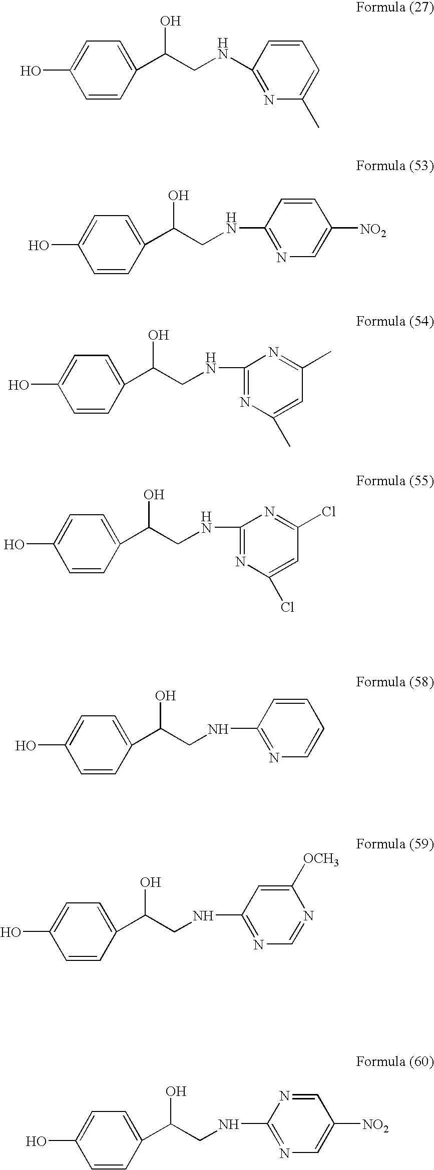
(j) a 5- or 6-membered non-aromatic heterocyclic ring having 1 or 2 heteroatoms independently selected from the group consisting of O, S, and N. The 5- or 6-membered non-aromatic heterocyclic ring may be independently unsubstituted or substituted with 1 to 4 substituents independently selected from item (k), below;
E.g., the Synephrine Derivatives represented by the formulae below: 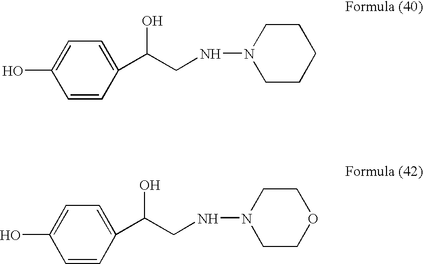
(k) In items (b) to (j), above, where there are substituents, the substituents are independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine.

In the case where R₁ and R₂ are bonded together, they are bonded together to include the N of Formula I to form a 5- or 6-membered heterocyclic ring. This N is counted as one heteroatom, an optional heteroatom may be included in the heterocyclic ring. The additional heteroatom is selected from the group consisting of O, S, and N. The heterocyclic ring may be independently unsubstituted or substituted with 1 to 4 substituents independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine.

E.g., the Synephrine Derivatives represented by the formulae below:

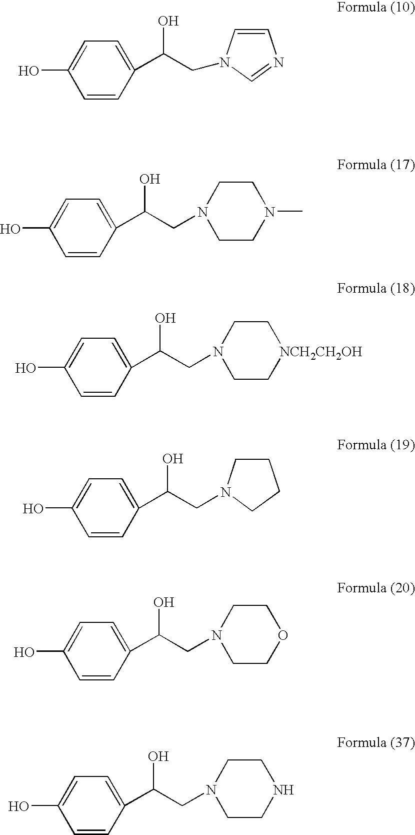

Non-limiting general examples of the Synephrine Derivatives are shown in Formulae G1 to G5, below, wherein “R” is selected from the group consisting of: H, hydroxy, halo, cyano, nitro, amino, phenyl, benzyl, CF₃, C₁ to C₆ alkyl, C₁ to C₆ alkoxy, C₁ to C₆ alkylol, and C₁ to C₆ alkylamine; and “Y” is selected from the group consisting of: CH, CH₂, N, NH, O, and S.

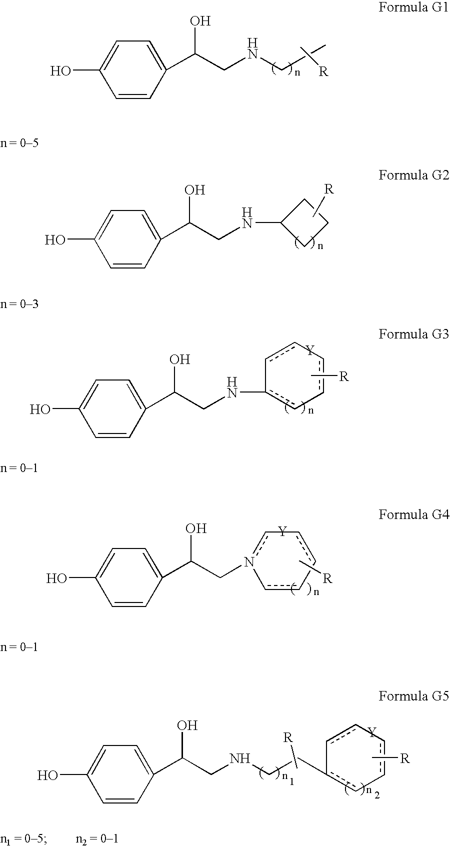

Specific examples of the Synephrine Derivatives are shown below:

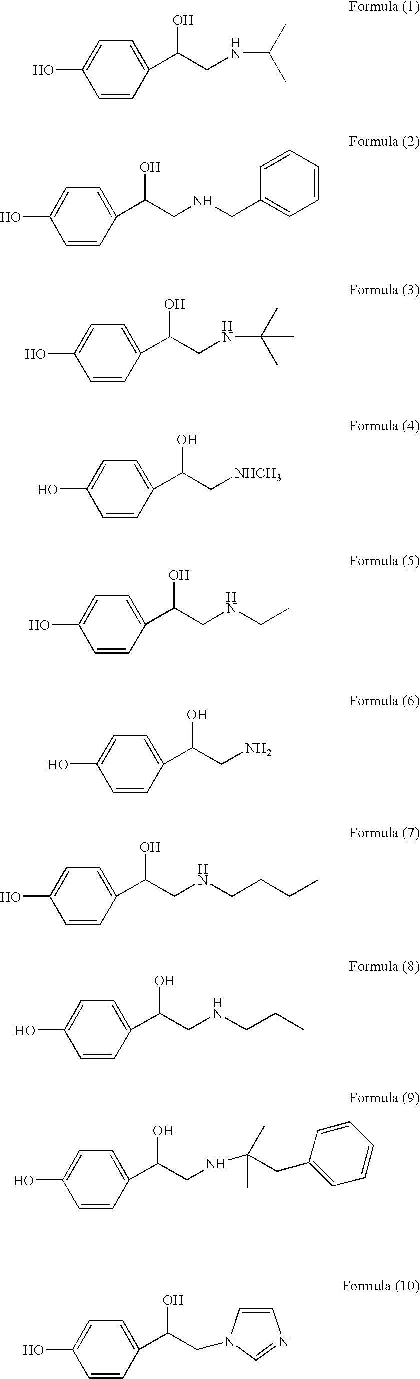

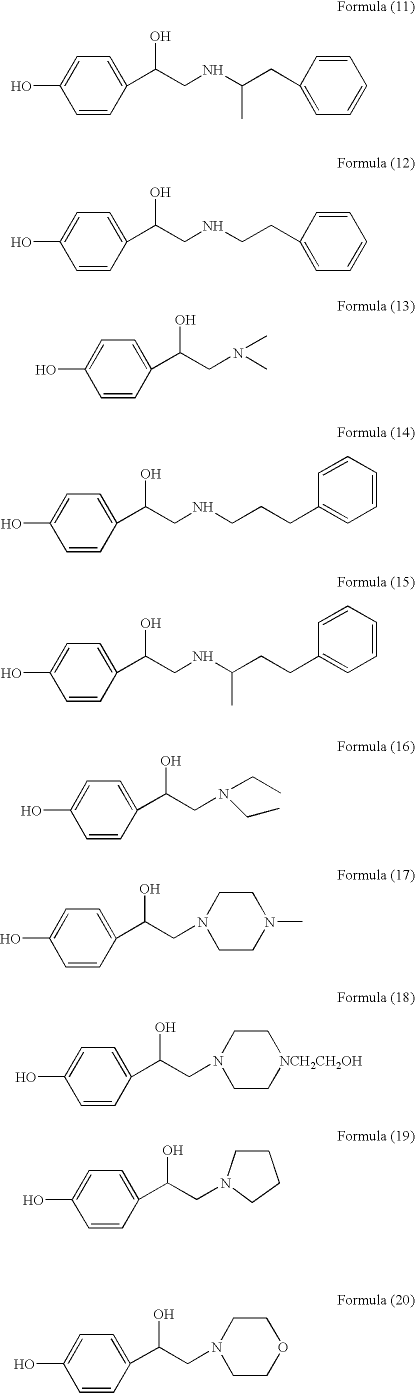

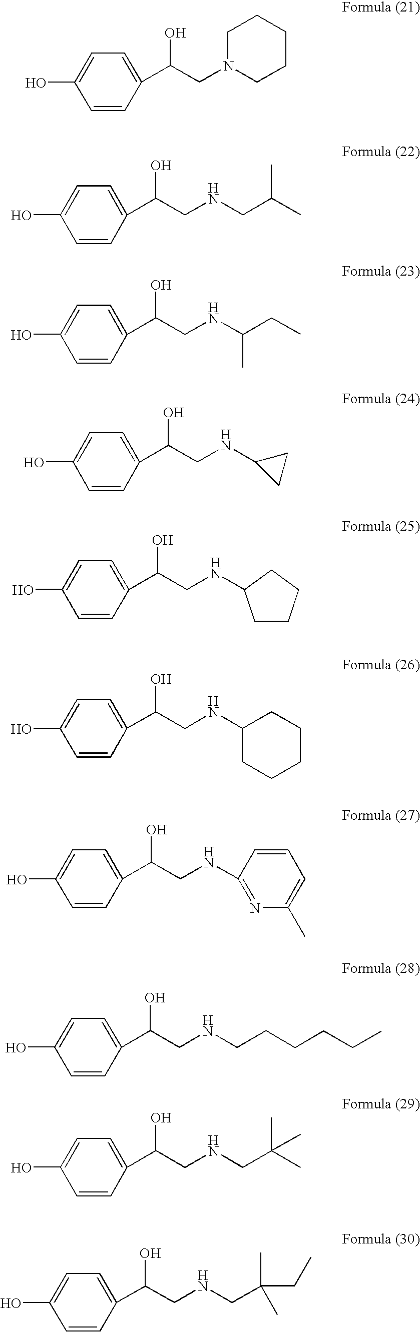

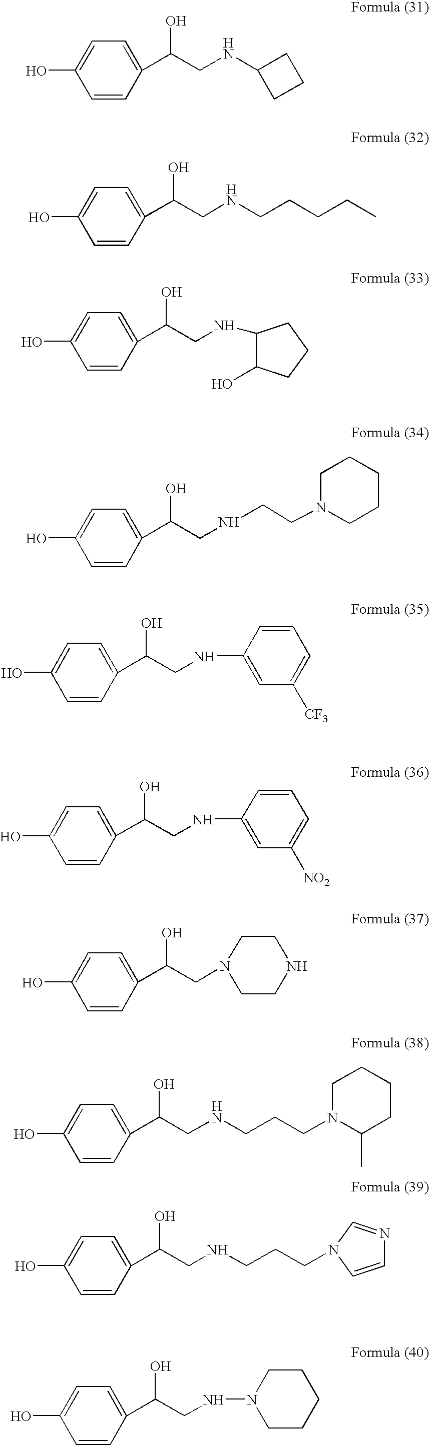

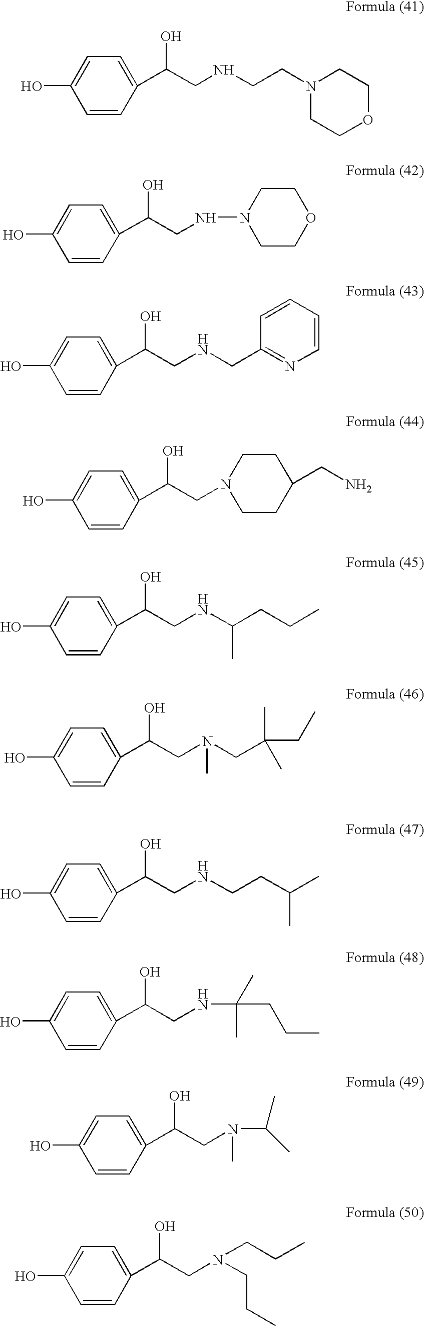

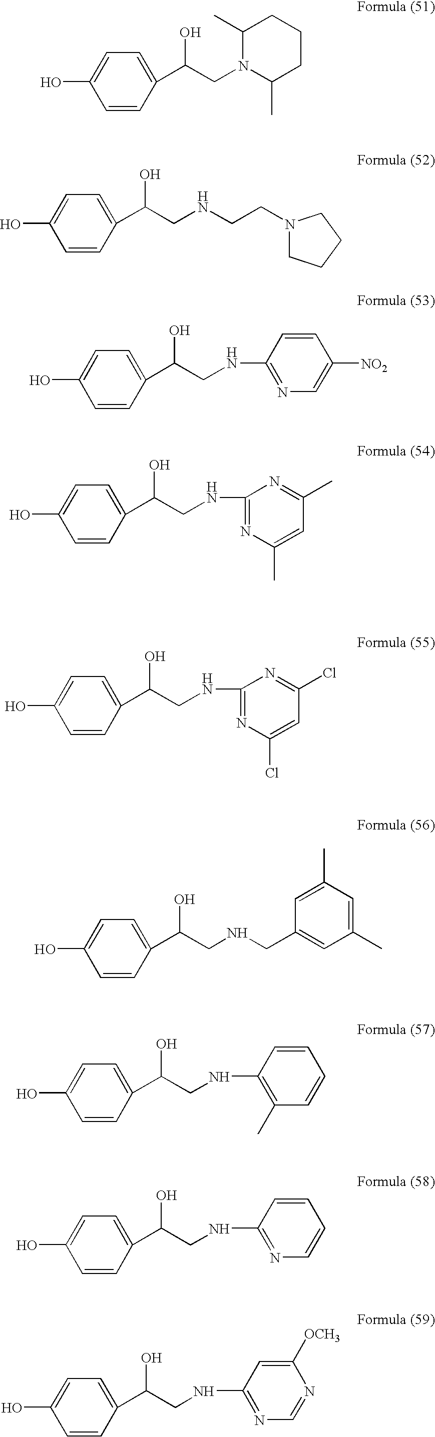

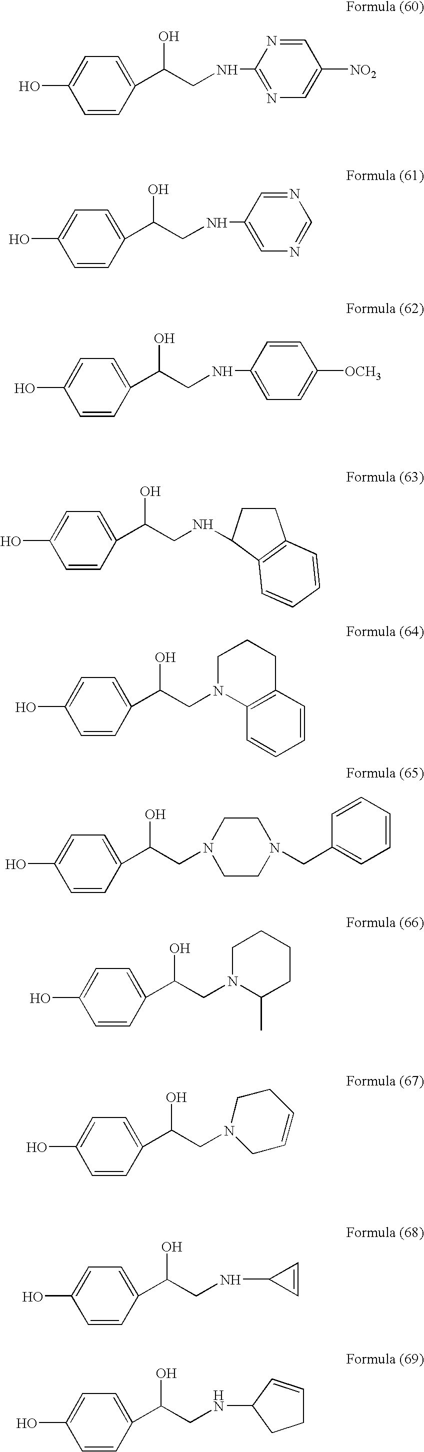

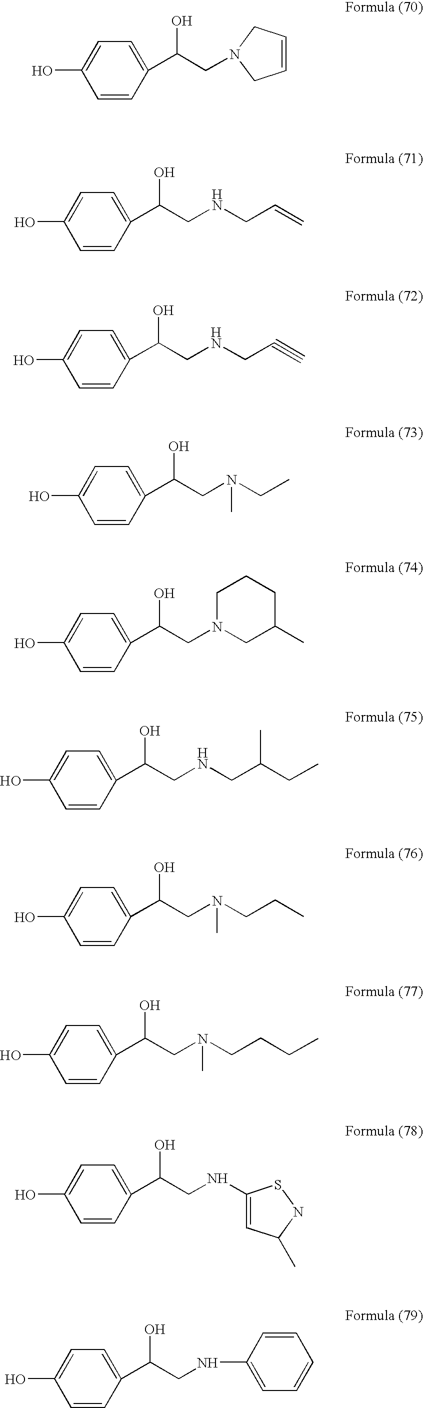

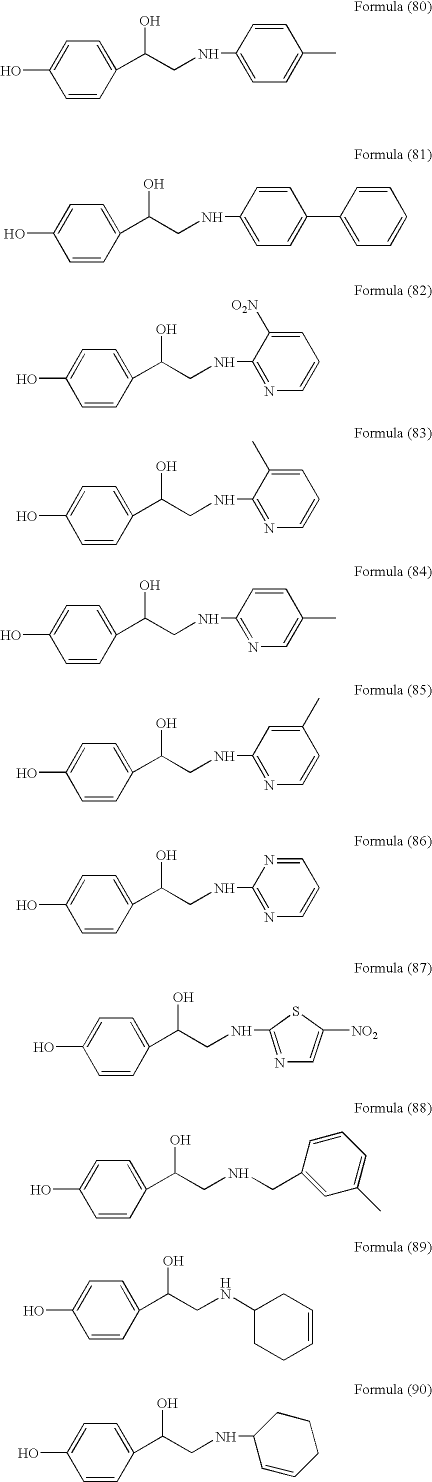

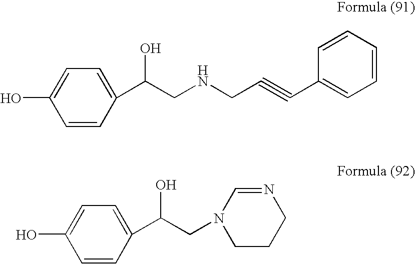

Applicant further believes that some of the Synephrine Derivatives disclosed herein are novel: such as Formula (28) to Formula (92), above.

Three-Step Synephrine Derivative Synthesis Method as Shown In Scheme 1.

The Synephrine Derivative is synthesized by a novel method which comprises the following three steps that are graphically shown in Scheme 1, below, wherein the R₁ and R₂ are as described for Formula I, above; and “X” is a halo (e.g., chloro, bromo, iodo and fluoro) substituent. Applicant believes that the following syntheses are also new: (1) the synthesis of the compound of Formula III from that of Formula II (that is, step 1 of Scheme 1); (2) the synthesis of the compound of Formula VI from that of Formula III; and/or (3) therefore, the synthesis of the compound of Formula VI from that of Formula II.

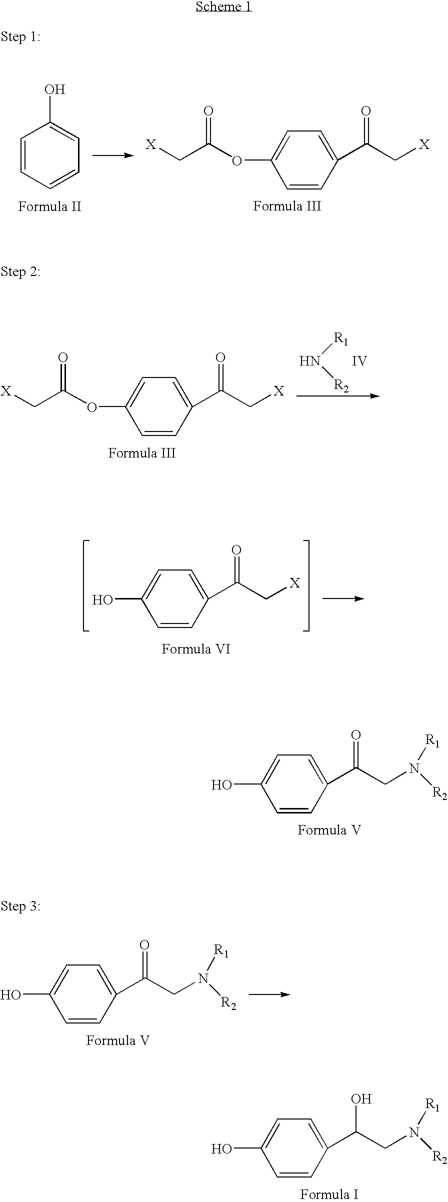

STEP 1: Through a reaction under Friedel-Crafts reaction condition, the phenol of Formula II and a haloacetyl halide, in the presence of a Lewis acid catalyst, solvent being optional, are reacted to produce a 2-halo-1-(4-haloacetyoxyphenyl)-ethanone intermediate, represented by Formula III, wherein X is a halo substituent.

In STEP 1, non-limiting examples of the haloacetyl halide are those having chloro, bromo, iodo or fluoro subtituent. An example of the haloacetyl halide is bromoacetyl chloride. The preferred haloacetyl halide is chloroacetyl chloride, which is relatively inexpensive. Preferably, the amount of haloacetyl halide used is from about 1.0 to about 6 moles. More preferably, the ratio of haloacetyl halide used is from about 1.1 to about 3 moles of haloacetyl halide per mole of phenol.

The catalyst which can be used is any Lewis acid known in the art. Non-limiting examples of Lewis acid which can be used are: aluminum chloride, aluminum bromide, zinc chloride, ferric chloride, stannic chloride, titanium chloride, and boron trifluoride. Preferably, aluminum chloride is used. The amount of Lewis acid is from 1 to 6 mole. Preferably, the amount is in the ratio of 2 to 3 moles of Lewis acid per mole of phenol.

STEP 1 can be realized in the absence of a solvent but the reaction proceeds more smoothly in an inert organic solvent. Non-limiting examples of the organic solvents are: sym-tetrachloroethane, dichloroethane, carbon disulfide, nitrobenzene, diethyl ether, dioxane. Preferably, dichloroethane is used. The solvent is preferably used in a volume of about 0.5 to about 10 times, more preferably the solvent is from about 0.5 to about 2 times, of the volume of the total reactants. Preferably, the reaction is carried out under anhydrous condition.

The reaction is preferably conducted at temperature from 0° C. to 100° C.; more preferably, the temperature is about room temperature to about 50° C. The reaction time is preferably from 0.5 hour to 20 hours, and more preferably from about 2 to about 6 hours. The reaction system is preferably from about 1 to about 2 atmospheric pressure.

STEP 2: The reaction intermediate [represented by Formula III of Scheme 1] is then mixed (solvent and basic catalyst being optional) with an amine [represented by Formula IV of Scheme 1], to produce a ketone intermediate [represented by Formula V of Scheme 1] in which their R₁ and R₂ are as defined above for Formula I.

In STEP 2, non-limiting examples of the amine which can be used are: alkylamines (non-limiting examples of which are methylamine, ethylamine, n-propylamine, iso-propylamine, n-butyl amine, sec-butylamine, tert-bultylamine); cycloalkylamines (non-limiting examples of which are cyclopropylamine, cyclobutylamine, cyclopentylamine, cyclohexylamine); aralkylamines (non-limiting examples of which are aniline, benzylamine, phenylethylamine, 3-methylbenzylamine, 3,5-dimethylbenzylamine); and substituted or unsubstituted heterocyclic amines (non-limiting examples of which are pyrrolidine, piperidine, morpholine, piperazine, 2-methylpiperidine, 3-methylpiperidine, N-methylpiperazine).

The reaction may be conducted in the absence of a catalyst. However, if one so wishes, an organic or an inorganic basic catalyst may be used. In some instances, the presence of an organic or an inorganic basic catalyst, makes the reaction smoother. Non-limiting examples of organic basic catalysts are: triethylamine, pyridine, and pyridazine. Non-limiting examples of inorganic basic catalysts are: ammonia, sodium hydroxide, potassium hydroxide, lithium hydroxide, sodium carbonate, and potassium carbonate.

The reaction between the reaction intermediate [represented by Formula III of Scheme 1] and the amine [represented by Formula IV of Scheme 1] can be carried out in the absence of a solvent since the amine reactant itself also serves as a solvent. However, it is preferable to conduct this reaction in a suitable solvent. Non-limiting examples of solvents which can be used in the reaction are: water; alcohols such as methanol, ethanol, and isopropanol; ethers such as dioxane, diethyl ether; esters such as ethyl acetate; aromatic hydrocarbons such as benzene, toluene, and xylene; nitrile solvents such as acetonitrils. Water, methanol and ethanol are preferred.

The reaction can be carried out with the compound of Formula IV being of the same molar amount or much larger molar amount than that of Formula III. Preferably, the compound of Formula IV is in a molar quantity of from about 2 to 20 times greater than that of Formula III.

The reaction temperature in this STEP 2 is from room temperature to a refluxing temperature, preferably from room temperature to about 100° C. The pressure in the reaction system is preferably from about atmospheric pressure to about 8 atmospheric.

STEP 3: The resulting ketone intermediates of Formula V of Scheme 1 are then reduced to obtain the Synephrine Derivatives. Non-limiting examples of the reducing methods are: reducing with hydrogen in the presence of a catalyst (e.g., using methods known in the art), reducing with a reducing agent. The catalyst used in the hydrogen reduction may be a non-chiral or a chiral catalyst (for the latter, see further discussion under the section, “Stereoisomeric Mixture and Pure Stereoisomers”, below).

The reduction by hydrogen can also be conducted in the presence of a catalyst. Non-limiting examples of such a catalyst are: palladium black, palladium-on-carbon, Raney nickel, platinum oxide, and platinum black. For example, the catalysts can be 5% or 10% palladium in active carbon, or can be active Raney nickel, or any catalysts known in the art for hydrogenation. The ratio of the amount of the catalyst to the ketone intermediate (Formula V of Scheme 1) is preferably from about 0.001 to about 5 moles, more preferably from about 0.01 to about 1 mole, per mole of the ketone intermediate.

Non-limiting examples of a reducing agent are: sodium borohydride and lithium aluminum hydride. The ratio of the reducing agent to the ketone intermediate (of Formula V) can be preferably from about 1 to about 10 moles, more preferably from about 2 to about 4 moles, per mole of the ketone intermediate.

Non-limiting examples of the reaction solvent are: water, methanol, ethanol, propanols and other alcohols. However, when lithium aluminum hydride is used as a reducing agent, the solvent is preferably a non-aqueous solvent, non-limiting examples of which are anhydrous diethyl ether, and tetrahydrofuran.

The reducing reaction temperature is preferably from about 0° C. to about 100° C., more preferably from about room temperature to about 50° C. For the catalytic reduction, the hydrogen pressure is from about atmospheric pressure to about 100 atmospheres, more preferably from about atmospheric pressure to about 50 atmospheres.

To illustrate the above method, the following is a non-limiting example for preparing the Synephrine Derivatives, starting from phenol:

This is a three-step synthetic route, starting from the cheap and commercially available materials: phenol and haloacetyl halide. STEP 1 is a reaction under Friedel-Crafts reaction condition, in which the phenol was condensed with haloacetyl halide, catalyzed by Lewis acid to obtain the novel intermediate 2-halo-1-(4-haloacetyoxyphenyl)-ethanone [represented by Formula III of Scheme 1, wherein X is a halo (e.g., chloro, bromo, iodo and fluoro) substituent]. In STEP 2, when 2-halo-1-(4-haloacetyoxyphenyl)-ethanone reacted with amine, the substantial reactant with amine was 2-halo-1-(4-hydroxy-pheny)-ethanone [represented by Formula VI of Scheme 1] which was first produced by easy hydrolysis of Formula III in situ in the reaction. Formula III served as the precursor of Formula VI in this step.

In one embodiment of the invention, to make the synthetic process easy to carry out, compound of Formula VI of Scheme 1 is generated in situ and is not isolated. Thus, compound of Formula III is directly converted to compound of Formula V. However, applicant has prepared compound of Formula VI through the hydrolysis of compound of Formula III, isolated and purified compound of Formula VI through recrystalization from methanol or any other alcohol known in the art capable of recrystalizing. For example, compound of Formula III may be reacted with a base to produce compound of Formula VI. Organic or inorganic bases may be used, non-limiting examples of which are NaOH, KOH, Na₂CO₃. An example of the reaction is shown in Example 3, below. Compound of Formula VI may then be reacted with an amine of Formula IV to produce compound of Formula V.

Non-limiting examples for preparations of 2-chloro-1-(4-chloroacetyloxyphenyl-ethanone [an example of Formula III of Scheme 1, wherein X is a chloro substituent] and 2-chloro-1-(4-hydroxyphenyl)-ethanone [an example of Formula VI of Scheme 1, wherein X is a chloro substituent] are described as follows.

2-chloro-1-(4-hydroxyphenyl) -ethanone prepared from phenol using the Friedel-Crafts reaction was reported by Wilds et al. [A. L. Wilds et al., “The Synthesis of 3-(p-Hyrdroxy phenyl)-cyclopentanone-1 and Related Compounds”, J. Am. Chem. Soc., 67: 286-290 (1945)] However, Wilds et al reported a product having a melting point between 147-151° C. Wilds et al reported only 40% yield of the product, after tedious multi-recrystallization from different mixture solvents. Ludwig et al., provided a process for separating 2-halo-1-(4-hydroxy phenyl)-ethanone and its corresponding regional isomer 2-halo-1-(2-hydroxyphenyl)-ethanone which were generated in the Friedel-Crafts reaction between phenol or anisole and haloacetyl halide. [Ludwig et al., U. S. Pat. No. 2,838,570, “Process of producing pure halogeno methyl (p-hydroxy phenyl)ketones”, issued on Jun. 10, 1958; and Ludwig et al., DE 935,363, issued on Nov. 17, 1955]. These two patents revealed that there is no region selectivity in the Friedel-Crafts reaction, and the separation process of isomers is relatively complicated, time-consuming, expensive and low yield. Gurjar et al., also reported the preparation of 2-chloro-1-(4-hydroxyphenyl)-ethanone from phenol by Friedel Crafts reaction, but their yield was only 50%. [M. K. Gurjar et al., “A New Route to (±)-Metoprolol”, Synthetic Communications, 20(22): 3489-3496 (1990)].

When the applicant followed the procedures of Wilds et al., and Gurjar et al., he obtained unsatisfactory results: black tar reaction mixture was obtained, TLC analysis indicated that the product's spot was very small, the tar mixture made the purification works very difficult, resulting in a very low yield of the product 2-chloro-1-(4-hydroxyphenyl)-ethanone.

Further, Wilds et al, supra.; Ludwig et al., U.S. Pat. No. 2,838,570, supra.; Ludwig et al., DE 935,363, supra; and Gurjar et al., supra., required high reaction temperatures (70° C.) for synthesizing 2-chloro-1-(4-hydroxyphenyl)-ethanone [the compound of Example 2]. Therefore, one skilled in the art would not have considered that a lower temperature would work for the reaction.

Applicant observed that the required high reaction temperature damaged the reaction and resulted in tar mixture. However, when applicant applied a lower temperature and changed the reaction conditions (resulting in the synthesis method disclosed herein), to applicant's surprise, the new synthesis method did not result in 2-chloro-1-(4-hydroxyphenyl)-ethanone [the compound of Example 2], but instead it resulted in 2-chloro-1-(4-chloroacetyoxylphenyl)-ethanone [the compound of Example 1] which is a novel compound. Applicant also noticed that the new synthesis method did not result in the undesirable tar mixture, and the working up process was simple and consisted of just pouring the reaction mixture into hydrochloride aqueous solution, filtration and recrystalization, and the yield was good. Applicant further devised the now synthesis method such that 2-chloro-1-(4-hydroxyphenyl)-ethanone [the compound of Example 2] was easily obtained by further hydrolysis of the 2-chloro-1-(4-chloroacetyoxylphenyl)-ethanone [the compound of Example 1] with basic condition, again with improved yield and without the tar mixture in the reaction process. Applicant's synthetic methods are applicable not just to producing chloro compound of the foregoing, but are generally applicable to producing any halo compounds of the foregoing by using the appropriate haloacetyl halide. That is, the “X” of Formulae III and VI of the synthetic pathway of Scheme 1, can be any halo substituent, such as chloro, bromo, iodo and fluoro.

In STEP 3, the resulting ketone intermediates (represented by Formula V) from step 2, are reduced to Synephrine Derivative (represented by Formula I).

For illustrative purposes, the reaction schemes depicted in Scheme 1 provides potential routes for synthesizing the Synephrine Derivatives as well as key intermediates, both as free bases and salts. For a more detailed description of the individual reaction steps, see the Examples section below, such as Examples 12 to 20 which present the methods for making some of the Synephrine Derivatives as free bases and salts. Those skilled in the art will appreciate that other synthetic routes may be used. Although specific starting materials and reagents are depicted herein, other starting materials and reagents can be easily substituted to provide various derivatives and/or reaction conditions. In addition, many of the compounds prepared by the methods described below can be further modified in light of this disclosure using conventional chemistry well known to those skilled in the art. Thus, based on the teaching of this application, one skilled in the art would be able to apply the synthesis methods presented herein, and if necessary to modify the synthesis methods using methods known in the art, to make the different Synephrine Derivatives.

Stereoisomeric Mixture and Pure Stereoisomers

The Synephrine Derivatives disclosed herein include both their stereoisomeric mixture and pure stereoisomers. The above method can produce stereoisomeric mixture. Pure stereoisomers can be obtained by resolution of the stereoisomeric mixture through well-known techniques such as the fractional precipitation of the diastereomeric salts formed with optically active acids, followed by conversion back to the pure stereoisomeric bases [Fabian, GB Pat. No. 816,857, “Resolution of Racemic Aminoalcohols”, specification published Jul. 22, 1959]. Pure stereoisomers can also be obtained by asymmetric hydrogenation catalyzed with chiral-catalysts [T. Ohkuma, et al., Asymmetric Hydrogenation, Ch. 1, Secs. 1.4.2 to 1.4.2.2, of Catalytic Asymmetric Synthesis, 2nd ed., I. Ojima, ed. (Wiley-VCH, John Wiley & Sons, Inc., Publication, New York, 2000)].

Use Of Synephrine Derivatives As Fat Breakdown Stimulators Or Body Weight Loss Agents

The Synephrine Derivatives can be classified as sympathomimetic derivatives. Some of the Synephrine Derivatives presented herein are known to possess pure β-adrenergic activity, some even specifically target only the β₃-receptor. For example, Anderson [W. G. Anderson, “The Sympathomimetic Activity of N-Isopropyloctopamine In Vitro”, J. Pharmacol. Exptl. Therap., 225(3): 553-558 (1983)] reported that N-isopropyloctopamine, possesses pure β-adrenergic activity. Carpene et al. [C. Carpene et al., “Selective activation of β₃-adrenoceptors by octopamine: comparative studies in mammalian fat cells”, Naunyn-Schmiedebergs Arch Pharmacol, 359: 310-321 (1999)] reported that octopamine can selectively act on β₃-adrenoceptors.

It is well established that β-adrenergic agonists are one of the most important stimulators of lipid-mobilization, especially it is said that β₃-adrenoceptor is solely responsible for lipolysis and thermogenesis. In contrast, α₂-adrenergic agonist inhibits lipolysis [S. W. Coppack, et al., “In vivo regulation of lipolysis in humans”, J. Lipid Res., 35:177-193 (1994)]. Thus, it appears that substances, such as synephrine, which possess both α- and β-adrenergic activity are surely not satisfactory agents for promoting fat breakdown and body weight loss. On the other hand, octopamine, another natural product without a substituted group on amino group, possesses selective activation of β₃-adrenoceptor. Unfortunately, octopamine is only effective in rat adipocyte lipolysis, it is ineffective with regard to human fat cells [C. Carpene et al., Naunyn-Schmiedebergs Arch Pharmacol, supra].

Despite the above, and the literature which discusses the derivatives of synephrine but does not disclose their uses or efficacies as fat breakdown stimulators or body weight loss agents in animals, applicant hypothesizes that by modifying the substituted groups on the nitrogen atom of the synephrine to produce the present Synephrine Derivatives, these Synephrine Derivatives would contain pure β-adrenergic or even only β₃-adrenergic property, which can stimulate lipid-mobilization and prompt fat breakdown or weight loss in animals, preferably mammals, most preferably in humans. Without wishing to be bound by the hypothesis, applicant further hypothesizes that Synephrine Derivatives which exhibit the property of adrenergic lipolysis in mammal and human adipose tissue in vitro, and which stimulate the increase of plasma free fatty acid level in vivo, are prime candidates as fat breakdown stimulators or body weight loss agents in animals. Thus, even though the scientific literature has not disclosed the uses of the Synephrine Derivatives as fat breakdown stimulators or body weight loss agents in animals, applicant is encouraged that some of the Synephrine Derivatives have been shown to exhibit the qualities applicant finds desirable for fat breakdown stimulators or body weight loss agents in animals. See, e.g., Pilkington, et al., Cernohorsky, et al., Muhlbachova, et al., and Wenkeova, et al., disclosed that synephrine [Formula (4), above], ethylnorsynephrine [Formula (5), above], propylnorsynephrine [Formula (8), above], isopropylnorsynephrine [Formula (1), above], Bamethan [Formula (7), above], tert-butylnorsynephrine [Formula (3), above], and phenyl-t-butylnorsynephrine [Formula (9), above] have lipid mobilization properties in vitro; isopropylnorsynephrine, Bamethan and synephrine can increase plasma free fatty acid level in vivo. [Moore et al, U.S. Pat. No. 2,460,144, supra.; T. R. E. Pilkington, et al., “Effect of sympathomimetic compounds with β-adrenergic effects on plasma free fatty acids in man”, J. Lipid. Res., 7(1): 73-76 (1966); M. Cernohorsky, et al., “The effects of some derivatives of noradrenaline and 2-mino-1-p-hydroxy-phenylethanol on the in vitro mobillisation of fat”, J. Pharm. Pharmac., 18:188-9 (1966); E. Muhlbachova, et al., “Lipid-Mobilizing Effects of Adrenaline, Noradrenaline, Isoproterenol and Isopropylnorsynephrine in Rabbit Adipose Tissue In Vivo” Physiologia Bohemoslovaca, 22(5): 503-510 (1973); J. Wenkeova, et al., “Adrenergic Lipolysis in Human Adipose Tissue In Vitro”, Eur. J. Pharmacology, 30(1):49-55 (1975).]

Applicant tested out and confirmed his hypothesis in Example 21 below, in which the tested isopropylnorsynephrine hydrochloride proved to be superior than synephrine hydrochloride in stimulating lipolysis of human fat cells. Furthermore, applicant notes that isopropylnorsynephrine and its salts have an advantage over the currently used synephrine in that isopropylnorsynephrine and its salts do not elevate blood pressure in in vivo test in rabbits [see, Muhlbachova, et al., supra, which tested isopropylnorsynephrine sulfate]. On the other hand, applicant notes that synephrine has long been used to increase blood pressure in China [X. W. Zhao, et al., “Anti-shock effects of synthetic effective compositions of fructus aurantii immaturus. Experimental study and clinical observation”, Chin. Med. J. (Engl), 102(2): 91-93 (1989)]. Since it is very dangerous to elevate blood pressure, especially for overweight people, applicant notes that drugs or dietary supplements with high efficiency for promoting weight loss, but with lower side effects or without side effects, are very important. Thus, a further aspect of the present invention presents the use of known or novel Synephrine Derivatives with little or no side-effect for promoting weight loss. Thus, in one embodiment of the invention, it is preferably to further test the Synephrine Derivatives for their effects on the physiology of the recipient, such as the blood pressure of the recipient, if such effects are not already known or reported. Tests known in the art may be used, such as those described in Muhlbachova et al., supra, or clinical tests for humans.

As shown above, some of the Synephrine Derivatives are known in the art, but some are novel. Thus, the present invention presents the new use of some known and some novel Synephrine Derivatives as fat breakdown stimulators or body weight loss agents in animals, preferably in mammals, and most preferably in humans. More generally, one aspect of the present invention presents a method for treating an animal having a disease, disorder or condition which can benefit from the Synephrine Derivatives which possess β-adrenergic or even only β₃-adrenergic property. The treatment step consists of administering to an animal or contacting the animal to an amount, preferably therapeutically effective amount, of a Synephrine Derivative or a combination of Synephrine Derivatives. Preferably, the foregoing are in the form of pharmaceutically acceptable compositions. The disease, disorder or condition known to benefit from compounds having β-adrenergic or even only β₃-adrenergic property, include obesity and diabetes. Based on the disclosure herein, one skilled in the art would also know that the following may be used besides the known or novel Synephrine Derivatives: solvates or hydrates of the Synephrine Derivatives; the prodrugs the Synephrine Derivatives, their salts, solvates and hydrates; as well as all stereoisomers (including diastereoisomers and enantiomers), tautomers and isotopically lablled of the foregoing. As explained previously, the compounds disclosed herein include both the pure stereoisomeric compounds and stereoisomeric mixtures. Preferably, the compounds are in the form of pharmaceutically acceptable compositions. Further details are provided below.

Non-Limiting Examples for Screening Synephrine Derivatives for Their Effects on Stimulating Fat breakdown

Further presented in this invention are methods for screening the Synephrine Derivatives to select for those which are fat breakdown or weight loss agents in animals, preferably in mammals, and most preferably in humans. As a start, applicant believes that Synephrine Derivatives which possess the property of adrenergic lipolysis in mammal and/or human adipose tissue in vitro, and which stimulates the increase of plasma free fatty acid level in vivo, are likely to be fat breakdown stimulators or body weight loss agents in animal, preferably mammals, and most preferably humans; and this ability could be further confirmed by screening using methods known in the art, including the methods disclosed in the present invention.

For convenience sake, in the following discussion, Synephrine Derivative is used as an example, even though as explained above, the following may bemused in the place of the Synpehrine Derivative: its solvates and hydrates, the prodrugs of the foregoing and the salts of the prodrugs, as well as all stereoisomers (including diastereoisomers and enantiomers), tautomers, and isotopically lablled of the foregoing. The compounds used may be pure stereoisomeric compounds, or stereoisomeric mixtures.

To facilitate the screening, confirmation, and efficacy of the Synephrine Derivatives as stimulators of fat breakdown or weight loss, the present invention also presents screening tests (which can be a screening test that is known in the art or the simple test shown in Examples 21 and 22 below) for selecting and confirming the Synephrine Derivatives that are useful as stimulators of fat breakdown or weight loss. The Synephrine Derivatives may further be screened for their ability or efficacy in causing fat breakdown or weigh loss using any method known in the art, such as disclosed in Carpene at al., Naunyn-Schmiedebergs Arch Pharmacol, supra. The efficacy of the Synephrine Derivatives to promote fat breakdown or weight loss can be measured by their ability to cause lipolysis in fat cells [e.g., in vitro tests as described in C. Carpene, “Assays of adrenergic receptors: including lipolysis and binding measurements”, from Methods in Molecular Biology, vol. 155: Adipose Tissue Protocols, Chapter 10, pp. 129-140, G. Ailhaud ed. (Humana Press Inc., Totowa, N.J., (2000))], their effect on blood plasma free fatty acid level of test subjects in in vivo test [A. Antonis, “Semiautomated method for the calorimetric determination of plasma free fatty acids”, J. Lipid. Res., 6(2): 307-312 (1965); Pilkington, et al., supra.] or their efficacy in causing weight loss in test subjects in clinical studies or trials [C. M. Colker, “Effects of citrus aurantium extracts, caffeine, and St. John's Wort on body fat loss, lipid levels, and mood states in overweight healthy adults”, Current Therapeutic Res., 60(3), 145-153 (1999)]. The step-by-step screening tests known in the art would enable one skilled the art to screen, select and confirm the efficacy of each Synephrine Derivative with regard to its ability to promote fat breakdown or weight loss.

As a non-limiting illustration of a screening test, for the test of lipolytic activation of the Synephrine Derivative, with regard to human adipocytes, dose-response of the Synephrine Derivative (on 5 log concentrations) can be studied by comparing the basal (without drugs) and maximal lipolysis (in the presence of a strandard reference e.g. isoproterenol, 10 μM)found in vitro.

Pharmacological and/or clinical tests known in the art [see, e.g., those described in Muhlbachova, et al., supra.; Antonis, supra.; Pilkington, et al., supra.; and Colker, supra] may be used to determine the Syneprhine Derivative's toxicity and efficacy, and the appropriate dose to be used in animals, preferably mammals, most preferably humans, to stimulate fat breakdown or weight loss. For example, Jones, U.S. Pat. No. 6,224,873B1, supra, also presents examples of clinical tests for weight loss, such as its Example 5, which may be modified for the present testing, e.g., by replacing Jones' compound with the present Synephrine Derivative. On the basis of pharmacologic and/or clinical test, one skilled in the art could arrive at an effective dose. For example, as a start, the Synephrine Derivative may be given at an effective dose of between about 0.01 mg and about 20 mg/kg, preferably between about 0.1 mg and about 10 mg/kg, most preferably between about 0.5 mg and about 5 mg/kg body weight per day. Depending on the results of the pharmacological and/or clinical tests, the foregoing doses may be adjusted upward or downward.

In clinical application, the dosage and dosage regimen must in each case be carefully adjusted, utilizing sound professional judgment and considering the age, weight and condition of the recipient, the route of administration and the nature and gravity of the disease, condition, or disorder. For general discussion of the foregoing, e.g., for an anti-obesity compound, see, e.g., Pfizer Prodcuts, Inc., PCT Publication WO 03/072572 A1, “Beta₃-Adrenergic Receptor Agosnists”, publ. Sep. 4, 2003; and Jones, U.S. Pat. No. 6,224,873B1, supra.

The administration can be by oral, buccal, transdermal, intradermal rectal, and parenteral (i.e. intramuscular, intravenous, and subcutaneous) routes. Generally, it will be found that when the Synephrine Derivative is administered orally, which is the preferred route, a larger quantity of reactive agent is required to produce the same effect as a smaller quantity given parenterally. In accordance with good clinical practice, it is preferred to administer the compounds at a concentration level that will produce effective fat breakdown effects or weight loss without causing any harmful or unwanted side effects.

The Synephrine Derivative can be given in the form of a pharmaceutical composition comprising an effective fat breakdown amount of the Synephrine Derivative. The following describes non-limiting examples of the pharmaceutical compositions and routes of administration or delivery. Other compositions and routes known in the art may be used to deliver the Synephrine Derivatives. Pharmaceutical compositions for effecting such results preferably contain at least about 0.5% to about 95% amount of a Synephrine Derivative in combination with a pharmaceutical carrier, the carrier comprising one or more solid, semi-solid, liquid or diluent, excipient, filler, lubricant, disintegrant, wetting agent and/or formulation adjuvant which is preferably non-toxic, inert and pharmaceutically acceptable. Such pharmaceutical compositions are preferably in dosage unit forms. A single dose preferably contains an amount sufficient to produce the desired fat breakdown effects. Pharmaceutical compositions which preferably provide from about 1.5 mg to about 1400 mg, more preferably from about 3.0 mg to about 700 mg, most preferably from about 5.0 mg to about 140 mg, of the active ingredient per unit dose are preferred and are conventionally prepared as tablets, coated tablets, lozenges, capsules, powders, transdermal patches; in diluents to form aqueous or oily suspensions, syrups, elixirs, and aqueous solutions. Preferred oral compositions are in the form of tablets or capsules and may contain conventional excipients such as binding agents (e.g. syrup, acacia, gelatin, sorbitol, or polyvinylpyrrolidone), fillers (e. g. lactose, sugar, maize-starch, calcium phosphate, sorbitol, or glycine), lubricants (e.g. magnesium stearate, talc, polyethylene glycol or silica), disintegrants (e.g. starch) and wetting agents (e.g. sodium lauryl sulfate). Suitable carriers (e.g. diluents and excipients) are well known to those skilled in the art and include materials such as carbohydrates, waxes, water soluble and/or swellable polymers, hydrophilic or hydrophobic materials, gelatin, oils, solvents, water, and the like. In general, for a carrier, safe solvents are non-toxic aqueous solvents such as water and other non-toxic solvents that are soluble or miscible in water. Suitable aqueous solvents include water, ethanol, propylene glycol, polyethylene glycols (e.g PEG400, PEG300), etc. and mixtures thereof. Solution or suspensions of a Synephrine Derivative with the usual pharmaceutical carriers are generally employed for parenteral compositions such as an aqueous solution for intravenous injection or an oily suspension for intramuscular injection. Such compositions having the desired clarity, stability and adaptability for parenteral use are preferably obtained by dissolving from 0.01% to 20%, more preferably from about 0.1% to about 10%, most preferably from about 0.5% to about 5%, by weight of the active Synephrine Derivative in water or a vehicle consisting of a polyhydric aliphatic alcohol such as glycerine, propyleneglycol, and polyethelene glycols or mixtures thereof. For weight loss or fat breakdown, the compositions may also include other anti-obesity agents such as MCR-4 agonists; and other pharmaceutical agents such as anti-depressants. Further details of other known pharmaceutical administrative routes, pharmaceutical ingredients, carriers, diluents, excipients, anti-obesity agents and other pharmaceutical agents which could be included in the present compositions and the types of composition (e.g., solid, liquid, suspension) may be used, such as disclosed in, e.g., Pfizer Prodcuts, Inc., PCT Publication WO 03/072572 A1, supra, and Jones, U.S. Pat. No. 6,224,873B1, supra.

The pharmaceutically acceptable compositions of the present invention may also include herbs and supplements. Further, the composition may contain one or more Synephrine Derivatives. Thus, the composition may also contain or be given in combination with other herbs that possess beneficial effects for animals, preferably mammals, and most preferably humans, and particularly in respect to weight loss or improvements in physical performance. In this connection, suitable herbs and foods include those herbs and foods that contain methylxanthines such as caffeine, theobromine and theophylline, which by virtue of their inhibition of the enzyme phosphodiesterase may potentiate the thermogenic actions of the Synphrine Derivative and increase the actions at the level of the β₃-receptors. At the same time, the actions of methylxanthines on α-receptors may serve to reduce or eliminate any unwanted cardiovascular effects, such as peripheral vasoconstriction and increase in blood pressure, that would be undesirable within the context of weight loss or improved physical performance. Suitable herbs and foods in this respect include, but are not limited to, Paullinia cupana (Guarana), Ilex paraguariensis (Mate), Cola nitida, Cola acuminata, Camellia sinensis (Tea), Coffea arabica (Coffee) and Theobroma cacao (Cocoa), whereby the herb or food may be used as the natural material or an extract thereof In such cases, the herb so chosen is admixed with the Synephrine Derivative in a suitable form to provide a solid or liquid dosage unit. See, e.g., Jones, U.S. Pat. No. 6,224,873B1, supra, which discloses further ingredients which may be added to the composition.

Yet another aspect of the invention presents a method for treating an animal having a disease, disorder or condition which can benefit from those Synephrine Derivatives which possess β-adrenergic or even only β₃-adrenergic property. Generally, these are diseases, conditions, or disorders modulated by β₃-adrenergic receptor agonists. The treatment step consists of administering to an animal or contacting the animal to a therapuetically effective amount of a compound of the present invention. The disease, disorder or condition known to benefit from compounds having β-adrenergic or even only β₃-adrenergic property, include weight loss, obesity, diabetes, irritable bowel syndrome, inflammatory bowel disease, esophagitis, duodenitis, Crohn's disease, proctitis, asthma, intestinal motility disorder, ulcer, gastritis, hypercholesterolemia, cardiovascular disease, urinary incontinence, depression, prostate disease, dyslipidemia, fatty liver, and airway inflammatory disorder. PCT Publication WO 03/072572 A1, supra, discloses the diseases, conditions, or disorders modulated by β₃-adrenergic agonists. Known clinical tests relevant to the disease of concern should be applied to determine doses, efficacy, etc.

The Synephrine Derivatives can also be extensively used as dietary and/or nutritional supplements in nutraceutical with the aim of fat breakdown or body weight loss. As effective fat breakdown ingredients, the Synephrine Derivatives can be used as food additives, such as those used in sweeteners, cakes, cookies, candy bars, nutrition bars, tea bags, etc. They can also be used in beverages such as tea, coffee, nutritional and sports drinks, and chewing gum; and in cosmetic products such as sunscreen, moisturizers, skin lotions, face/body creams, lipsticks, preferably at an amount which will produce effective fat breakdown effects without causing any harmful or unwanted side effects. Jones, U.S. Pat. No. (6,224,873 B1. supra, Example 1, discloses the use of its weight loss agent in tea bags, the method for determining the doses, and having human subjects drink the tea and recording their reactions, such as their levels of hunger and energy. This method may be applied to the present invention by replacing Jones' active ingredient with the Synephrine Derivatives. The above are non-limiting examples of the uses, compositions, and methods, such as for determining the efficacy, toxicity, dosages, of the Synephrine Derivatives. Other uses, compositions, and methods apparent to one skilled in the art are also included in the scope of the present invention.

The invention is further exemplified and illustrated by the following examples, but these examples are given for illustrative purpose only and are not to be construed as limiting the scope of the invention.

### EXAMPLES

### Example 1

### The Novel Synthesis of the Novel 2-Chloro-1-(4-chloroacetoxylphenyl)-ethanone [an example of Formula III, wherein X is a chloro substituent]

This example presents the synthesis of the novel intermediate 2-chloro-1-(4-chloroacetoxylphenyl)-ethanone represented by the formula below:

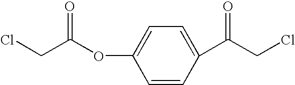

9.4 g of phenol was dissolved in 10 ml of dichloroethane, the solution was cooled in an ice bath. 33.4 g aluminum chloride powder was added slowly with stirring to the phenol solution, the ice bath was removed when the addition was finished. 25.0 g of chloroacetyl chloride in 15 ml of dichloroethane was added drop by drop over a period of almost 1 hour. A lot of hydrochloride was generated. After the reaction mixture had been stirred for 5 hours at 25° C., it was hydrolyzed with hydrochloric acid and ice. Insoluble products was filtered out and washed thoroughly with dilute acid and water. After drying, the pale yellow product of 23.1 g was obtained, yield 94%, mp 105-106° C., ¹HNMR (CDCl₃, ppm): σ 4.34 (s, 2H), 4.69 (s, 2H), 7.30 (d, 2H), 8.04 (d, 2H). IR (KBr): 1655 cm⁻¹, 1670 cm⁻¹. MS: M⁺246.

### Example 2

### The Prior Art Synthesis of the Known 2-Chloro-1-(4-hydroxyphenyl)-ethanone [an example of Formula VI, wherein X is a chloro substituent]

This example presents the prior art for the synthesis of 2-chloro-1-(4-hydroxyphenyl)-ethanone represented by the formula below:

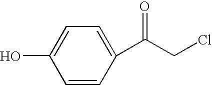

9.4 g of phenol was dissolved in 10 ml of dichloroethane, the solution was cooled in an ice bath. 33.4 g aluminum chloride powder was added slowly with stirring to the phenol solution, the ice bath was removed when the addition was finished. 17.0 g of chloroacetyl chloride in 15 ml of dichloroethane was added drop by drop over a period of almost 1 hour. A lot of hydrochloride was generated. After the reaction mixture had been stirred for 12 hours at 65° C., it was hydrolyzed with hydrochloric acid and ice. Insoluble substance was extracted with dichloromethane, solution was dried by anhydrous soldium sulfate, purified by SiO₂ column chromatograph, crystallized with methanol to give white crystal 2.25 g, yield 15%, mp 151-152° C., ¹HNMR (CDCl₃, ppm): σ 4.66 (s, 2H), 6.92 (d, 2H), 7.92 (d, 2H). IR (KBr): 1650 cm⁻¹.

### Example 3

### A Novel Synthesis of the Known 2-Chloro-1-(4-hydroxyphenyl)-ethanone by Hydrolysis [an example of Formula VI, wherein X is a chloro substituent]

This example presents the a novel synthesis of the known 2-chloro-1-(4-hydroxyphenyl)-ethanone [formula shown in Example 2, above].

10 g of 2-chloro-1-(4-chloroacetoxylphenyl)-ethanone from Example 1 was suspended in 50 ml methanol, the suspension was cooled in an ice-water bath. Equivalent amount of 5% aqueous sodium hydroxide was added drop by drop. After the addition was completed, the mixture was stirred at room temperature for 0.5 hour. The proper amount of reaction solvent was distilled, the residue mixture was cooled, crystals were formed, then through filtration, 5.9 g crystal was obtained, the yield was 85%, the mp was 151-152° C.

### Example 4

### The Novel Synthesis of the Known 2-Isopropyl amino-1-(4-hydroxyphenyl)-ethanone Hydrochloride [an example of the salt of Formula V]

2-Isopropylamino-1-(4-hydroxyphenyl)-ethanone hydrochloride is represented by the formula below:

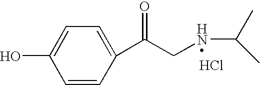

1.0 g of 2-chloro-1-(4-chloroacetoxylphenyl)-ethanone [Formula shown in Example 1] was suspended in 10 ml of methanol, 5 ml of isopropylamine was added drop by drop, the suspension was dissolved gradually, the resulting solution was stirred at room temperature for 6 hours, excess isopropylamine was removed, the resulting solution was acidified with dilute hydrochloride, then evaporated to dry, the residue was crystallized from methanol to produce the product 0.67 g, yield 72%, mp 256-257° C., ¹HNMR (D₂O, ppm): σ 1.22 (d, 6H), 3.38 (m, 1H), 4.54 (s, 2H), 6.83 (d, 2H), 7.77 (d, 2H). IR (KBr) : 1655 cm⁻¹, 3320 cm⁻¹.

### Example 5

### A Novel Alternative Synthesis of the Known 2-Isopropylamino-1-(4-hydroxyphenyl)-ethanone Hydrochloride [an example of the salt of Formula V]

2-Isopropylamino-1-(4-hydroxyphenyl)-ethanone hydrochloride [formula shown in Example 4, above] may also be synthesized as follows: 1.0 g of 2-chloro-1-(4-hydroxyphenyl)-ethanone [formula shown in Example 2, above. This is an example of Formula VI] was dissolved in 10 mL methanol, 5 ml of isopropylamine was added dropwise while stirring. After the mixture had been stirred for 3 hours at 20° C., excess isopropylamine was removed by distillation. Diluted hydrochloride was added to acidify the residue. The mixture was evaporated to dryness, the residue was crystallized from methanol to obtain colorless product 1.0 g, yield 75%, mp 257-258° C.

### Example 6

### The Novel Synthesis of the Known 2-Methyl amino-1-(4-hydroxyphenyl)-ethanone Hydrochloride [an example of the salt of Formula V]

2-Methylamino-1-(4-hydroxyphenyl) -ethanone hydrochloride is represented by the formula below:

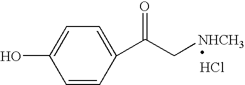

1.0 g of 2-chloro-1-(4-chloroacetoxylphenyl)-ethanone [formula shown in Example 1, this is an example of Formula III] was dissolved in 10 mL methanol, 20 mL of 40% methylamine aqueous was added drop by drop in 10 mins, the reaction solution was stirred at room temperature 4 hours, then excessive methylamine was recycled, solid residue was recrystallized from ethanol-HCl aqueous to obtain crystal 0.47 g, mp 141° C. (dec.), yield 71%.

### Example 7

### The Novel Synthesis of the Known 2-Ethyl amino-1-(4-hydroxyphenyl)-ethanone Hydrochloride [an example of the salt of Formula V]

2-Ethylamino-1-(4-hydroxyphenyl)-ethanone hydrochloride is represented by the formula below:

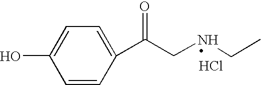

2-Ethylamino-1-(4-hydroxyphenyl)-ethanone hydrochloride may be synthesized by replacing the isopropylamine of Example 4, above, with aqueous ethylamine.

### Example 8

### The Novel Synthesis of the Known 2-tert-Butyl amino-1-(4-hydroxyphenyl)-ethanone Hydrochloride [an example of the salt of Formula V]

2-tert-Butylamino-1-(4-hydroxyphenyl)-ethanone hydrochloride is represented by the formula below:

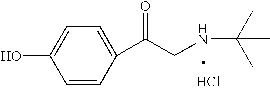

2-tert-Butylamino-1-(4-hydroxyphenyl)-ethanone hydrochloride may be synthesized by replacing the isopropylamine of Example 4, above, with aqueous tert-butylamine.

### Example 9

### The Novel Synthesis of the Known 2-Benzyl amino-1-(4-hydroxyphenyl)-ethanone Hydrochloride [an example of the salt of Formula V]

2-Benzylamino-1-(4-hydroxyphenyl)-ethanone hydrochloride is represented by the formula below:

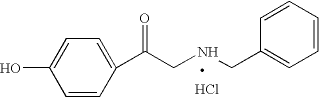

2-Benzylamino-1-(4-hydroxyphenyl)-ethanone hydrochloride was synthesized as follows: 1 g of 2-chloro-1-(4-chloroacetoxyl phenyl)-ethanone [formula shown in Example 1, above] was dissolved in 10 mL benzylamine, the solution was stirred at room temperature 1 hour, 100 mL hexane was added, the precipitate was collected by filtration, and then dissolved in 10% HCl 10 mL, insoluble substance was filtrated, the solution was evaporated to dryness, the residue was crystallized from methanol, 0.66 g crystal obtained, yield 68%, mp: 245-236° C.

### Example 10

### The Novel Synthesis of the Novel 2-(2-Hydroxyl cyclopentylamino)-1-(4-hydroxyhenyl)-ethanone [an example of Formula V]

2-(2-Hydroxylcyclopentylamino)-1-(4-hydroxyhenyl)-thanone is represented by the formula below:

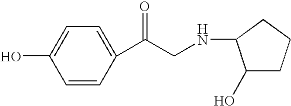

2-(2-Hydroxylcyclopentylamino)-1-(4-hydroxyhenyl)-thanone may be synthesized as follows: 1.0 g of 2-aminocyclopentanol hydrochloride is dissolved in 25 ml methanol, 1.2 g of 2-chloro-1-(4-chloroacetoxylphenyl)-ethanone [formula shown in Example 1, above] and 5 ml of triethylamine are added to the methanol solution. The reaction mixture is stirred at room temperature for 5 hours, then evaporated to dryness; after which 25 mL of water is added, the resulting solution is extracted with chloroform, and the chloroform solution is then washed with water. The washed chloroform solution is dried with anhydrous sodium sulfate, then the mixture is filtrated, the filtrate is evaporated to dryness to obtain solid residue. The residue is recrystalized from methanol to obtain the crystal of 2-(2-hydroxylcyclopentylamino)-1-(4-hydroxyhenyl)-ethanone.

### Example 11

### The Novel Synthesis of the Novel 2-Piperazine-1-(4-hydroxyphenyl)-ethanone Sulfate [an example of the salt of Formula V]

2-Piperazine-1-(4-hydroxyphenyl)-ethanone sulfate, is represented by the formula below:

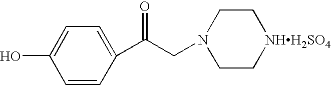

2-Piperazine-1-(4-hydroxyphenyl) -ethanone sulfate may be synthesized as follow: 10.0 g of piperazine hexahydrate is dissolved in 35 ml methanol, 1.0 g of 2-chloro-1-(4-chloroacetoxylphenyl)-ethanone [formula shown in Example 1, above] is added to the methanol solution. The reaction mixture is stirred at room temperature for 6 hours. The reaction mixture is then evaporated in vacuum at 80° C. to remove methanol and excess piperazine hexahydrate. The residue is suspended in water, suitable amount of 5% sulfuric acid aqueous is added to adjust the solution to pH5, the solution is then evaporated to dryness to obtain the solid residue. The residue is recrystallized from methanol to obtain the crystal of 2-piperazine-1-(4-hydroxyphenyl)-ethanone sulfate.

### Example 12

### The Prior Art Synthesis of the Known 2-Isopropylamino-1-(4-hydroxyphenyl)-ethanol Hydrochloride, Also Known as Isopropylnorsynephrine Hydrochloride [an example of the salt of Formula I]

2-Isopropylamino-1-(4-hydroxyphenyl)-ethanol hydrochloride is represented by the formula below [This is the hydrochloride salt of Formula (1)]:

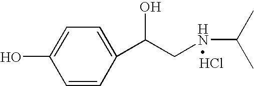

1.0 g of 2-isopropylamino-1-(4-hydroxyphenyl)-ethanone hydrochloride [formula shown in Example 4, above] was dissolved in 15 ml H₂O, 50 mg of 5% palladium on active carbon was added to the resulting solution. Hydrogen was introduced after the air was removed from the reactor. The reaction mixture was stirred at room temperature for 8 hours under 3 atmospheric pressures. After the completion of the reduction reaction, the catalyst was filtered, and the filtrate was concentrated to dryness. The residue was crystallized from methanol to obtain colorless crystals 0.90 g, yield 90%. mp 151.5-152.5° C. ¹HNMR (D₂O, ppm): σ 1.47 (d, 6H), 3.08 (d, 2H), 3.30 (m, 1H), 4.75 (t, 1H), 6.74 (d, 2H), 7.13 (d, 2H). IR (KBr): 3305 cm⁻¹

### Example 13

### The Prior Art Alternative Synthesis of the Known 2-Isopropylamino-1-(4-hydroxyphenyl)-ethanol Hydrochloride [an example of the salt of Formula I]

Example 12 presents the formula of 2-isopropylamino-1-(4-hydroxyphenyl)-ethanol hydrochloride. This example presents the alternative method for synthesizing the same compound.

2-Isopropylamino-1-(4-hydroxyphenyl)-ethanol hydrochloride was synthesized as follows: 1.0 g of 2-isopropyl amino-1-(4-hydroxyphenyl)-ethanone hydrochloride [formula shown in Example 4, above] was dissolved in 15ml methanol, 50 mg of 10% palladium on active carbon was added to the resulting solution. Hydrogen was introduced after the air in the reactor removed. The reaction mixture was stirred at room temperature for 24 hours under atmospheric pressure. Upon the completion of the reduction reaction, the catalyst was filtered, and the filtrate was concentrated to dryness. The residue was crystallized from methanol to obtain colorless crystals 0.91 g, yield 91%. mp 152-152.5° C.

### Example 14

### Novel Alternative Synthesis of the Known 2-Isopropyl amino-1-(4-hydroxyphenyl)-ethanol Hydrochloride [an example of the salt of Formula I]

Example 12 presents the formula of 2-isopropylamino-1-(4-hydroxyphenyl)-ethanol hydrochloride. This example presents the alternative method for synthesizing the same compound.

2-Isopropylamino-1-(4-hydroxyphenyl)-ethanol hydrochloride may also be synthesized as follows: 1.0 g of 2-isopropylamino-1-(4-hydroxyphenyl)-ethanone hydrochloride [formula shown in Example 4, above] is dissolved in methanol 10 ml, 0.5 g of sodium borohydride is added to the solution, the mixture is stirred for 2 hour at room temperature. Dilute hydrochloric acid is added to adjust to pH 2 to 3, the mixture is filtered, the filtrate is concentrated, then placed in a refrigerator to allow for crystallization, the crystal is recrystallized from methanol to obtain the final product which is expected to be colorless and to have a mp of from 151-152° C.

### Example 15

### The Prior Art Synthesis of the Known 2-Methyl amino-1-(4-hydroxyphenyl)-ethanol Hydrochloride [an example of the salt of Formula I]

2-Methylamino-1-(4-hydroxyphenyl)-ethanol hydrochloride, is represented by the formula below [which is the hydrochloride salt of Formula (4)]:

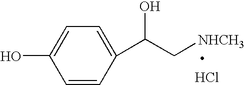

Using the method shown in Example 13, 2-methylamino-1-(4-hydroxyphenyl)-ethanone hydrochloride [formula shown in Example 6, above] was reduced to produce 2-methylamino-1-(4-hydroxyphenyl)-ethanol hydrochloride. Yield 89%. mp 185-180° C. ¹HNMR (D₂O, ppm): σ 62.61 (5, 3H), 3.12 (d, 2H), 4.81 (m, 1H), 6.77 (d, 2H), 7.15 (d, 2H), IR (KBr): 3311 cm⁻¹. Alternatively, in the foregoing, the method of either Example 12 or Example 14 may be used instead of that of Example 13, to produce 2-methylamino-1-(4-hydroxyphenyl)-ethanol hydrochloride.

### Example 16

### The Synthesis of the Known 2-Ethylamino-1-(4-hydroxyphenyl)-ethanol Hydrochloride [an example of the salt of Formula I]

2-Ethylamino-1-(4-hydroxyphenyl)-ethanol hydrochloride, is represented by the formula below [which is the hydrochloride salt of Formula (5)]:

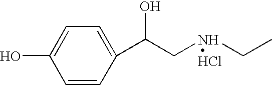

Using the method shown in Example 12, 13 or 14, 2-ethylamino-1-(4-hydroxyphenyl)-ethanone hydrochloride [formula shown in Example 7, above] may be reduced to produce 2-ethylamino-1-(4-hydroxyphenyl)-ethanol hydrochloride.

### Example 17

### The Synthesis of Known 2-tert-Butylamino-1-(4-hydroxyphenyl)-ethanol Hydrochloride [an example of the salt of Formula I]

2-tert-Butylamino-1-(4-hydroxyphenyl)-ethanol hydrochloride is represented by the formula below [which is the hydrochloride salt of Formula (3)]:

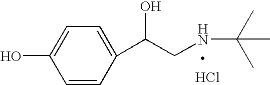

Using the method shown in Example 12, 13 or 14, 2-tert-butylamino-1-(4-hydroxyphenyl)-ethanone hydrochloride [formula shown in Example 8, above] may be reduced to produce 2-tert-butylamino-1-(4-hydroxyphenyl)-ethanol hydrochloride.

### Example 18

### Novel Synthesis of the Known 2-Benzyl amino-1-(4-hydroxyphenyl)-ethanol Hydrochloride [an example of the salt of Formula I]

2-Benzylamino-1-(4-hydroxyphenyl)-ethanol hydrochloride, is represented by the formula below [which is the hydrochloride salt of Formula (2)]:

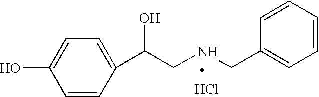

2-Benzylamino-1-(4-hydroxyphenyl)-ethanol may be synthesized as follows: 1.0 g of 2-benzylamino-1-(4-hydroxyphenyl)-ethanone hydrochloride [formula shown in Example 9, above] is dissolved in methanol 10 ml, 0.45 g of sodium borohydride is added to the solution, the mixture is stirred for 3 hours at room temperature. Dilute hydrochloric acid is added to adjust the solution to a pH of from 2 to 3, the mixture is filtrated, the filtrate is concentrated, the residue is placed in a refrigerator to let it solidify, the crystal is recrystallized from methanol to obtain the final product which is expected to be colorless and to have a mp of between from 161-162° C.

### Example 19

### Novel Synthesis of the Novel 2-(2-Hydroxyl cyclopentylamino)-1-(4-hydroxyphenyl)-ethanol [an example of Formula I]

2-(2-Hydroxylcyclopentylamino)-1-(4-hydroxyphenyl)-ethanol is represented by the formula below [Formula (33)].

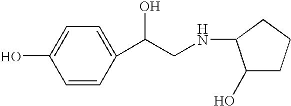

Using the same method as shown in Examples 12, 13 or 14, 2-(2-hydroxylcyclopentylamino)-1-(4-hydroxyphenyl)-ethanone [formula shown in Example 10, above] may be reduced to produce 2-(2-hydroxylcyclopentylamino)-1-(4-hydroxyphenyl)-ethanol.

### Example 20

### Novel Synthesis of Novel 2-Piperazine-1-(4-hydroxy phenyl)-ethanol Sulfate [an example of the salt of Formula I]

2-Piperazine-1-(4-hydroxyphenyl)-ethanol sulfate is represented by the formula below [which is a sulfate of Formula (37)]:

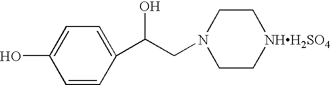

Using the method shown in Example 12, 13 or 14, 2-piperazine-1-(4-hydroxyphenyl)-ethanone sulfate [formula shown in Example 11, above] may be reduced to produce 2-piperazine-1-(4-hydroxyphenyl)-ethanol sulfate

### Example 21

### Test of lipolytic capacity of a Synephrine Derivative on Human Adipocytes

The lipolytic capacity of the Synephrine Derivatives can be tested in human adipocytes. For the sake of convenience, for Examples 21 to 24, the known 2-isopropylamino-1-(4-hydroxyphenyl)-ethanol hydrochloride (Formula shown in Example 12, above, also known as isopropylnorsynephrine hydrochloride) is used here as a non-limiting example of a Synephrine Derivative. Other Synephrine Derivatives may be used, non-limiting examples of which are: ethylnorsynephrine [Formula (5), above], propylnorsynephrine [Formula (8), above], Bamethan [Formula (7), above], tert-butyl-norsynephrine [Formula (3), above], and phenyl-t-butylnorsynephrine [Formula (9), above], tert-butylnorsynephrine hydrochloride [its free base formula is shown in Formula (3), above], bamethan hydrochloride [its free base formula is shown in Formula (7), above], and propylnorsynephrine hydrochloride[its free base formula is shown in Formula (8), above]. Formula of bamethan hydrochloride, tert-butylnorsynephrine hydrochloride, and propylnorsynephrine hydrochloride are shown below:

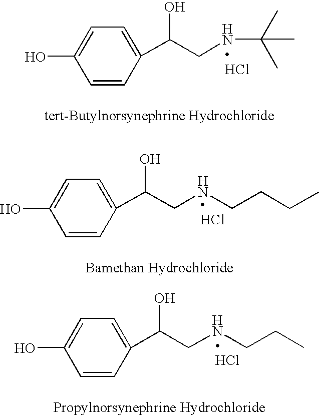

In this example, the lipolytic property of isopropylnorsynephrine hydrochloride was compared to that of synephrine hydrochloride (Formula shown in Example 15). The index of lipolytic activity chosen was the glycerol released by freshly originated human adipocytes with reference to basal (0%, without any drug) and maximal (100%) responses to a standard lipolytic reference: isoproterenol.

Adipocytes were isolated from samples of subcutaneous abdominal adipose depot freshly obtained during abdominal lipectomy. The surgical interventions were conducted under general anaesthesia on ten healthy women undergoing abdominal plastic surgery. The human samples were transferred in less than 15 min to the laboratory for adipocyte preparation. The human abdominal subcutaneous adipocytes were obtained by collagenase digestion of the fresh samples of adipose tissue. The isolation, washing, handling and distribution of fat cells were carried out according to the technique described in Carpene, “Assays of Adrenergic Receptors: Including Lipolysis and Binding Measurements”, from Methods in Molecular Biology, supra. Briefly, extemporaneous dilutions of the tested drugs were added in 4 μl in plastic vials which received 400 μl of freshly prepared adipocyte suspension. The given concentrations were the final concentrations in which the cells were incubated for 90 min at 37° C. under gentle shaking. At the end of the experiment, the glycerol released by the cells was determined on an aliquot as described in Carpene, Id.

The mean lipid content in the incubation vials was 17.6±1.0 mg/400 μl, which corresponded to approximately 500,000 cells/vial.

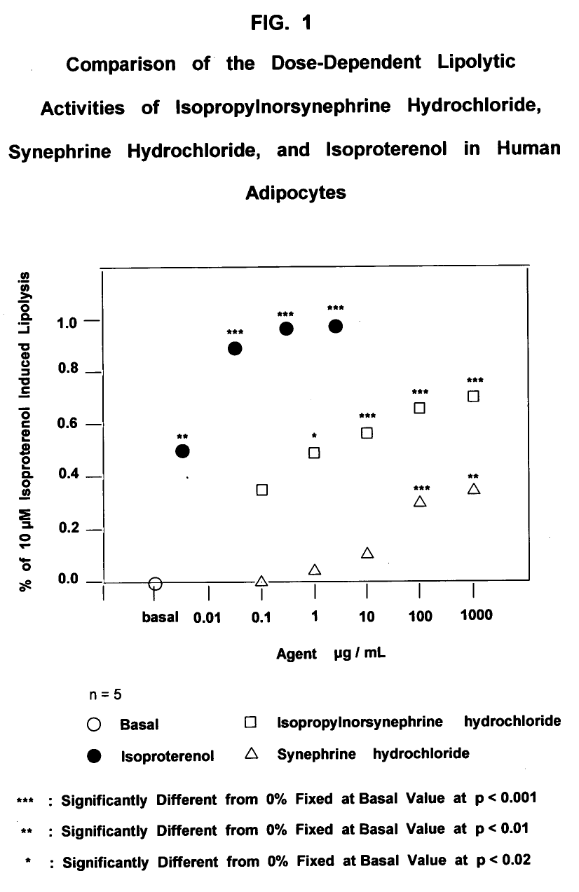

The result was shown in FIG. 1 which shows that isopropylnorsynephrine hydrochloride possessed a strong lipolytic property, it stimulated the lipolysis of human fat cell with high efficacy. Compared to synephrine hydrochloride, isopropylnorsynephrine hydrochloride had 2 times the capacity of synephrine hydrochloride to stimulate human adipocytes lipolysis, it elicited almost 60% of the maximal lipolytic response (synephrine hydrochloride elicited almost 30% of the maximal lipolytic response). Further, the affinity of isopropylnorsynephrine hydrochloride to adipocyte was almost 100 times that of synephrine hydrochloride. When the concentration was as low as 1μg/mL, the lipolytic effect of isopropylnorsynephrine hydrochloride was still far greater than that of synephrine hydrochloride which was at the concentration of 100 μg/mL. Thus, the present invention presents the new use of the known compound isopropylnorsynephrine hydrochloride as an ideal Synephrine Derivative which is useful for fat breakdown or weight loss in mammals, especially in humans.

### Example 22

### Non-Limiting Example of a Tablet Formulation

The Synephrine Derivatives may be formulated in tablet form as shown in Table 1, below. For the sake of convenience, the known compound isopropylnorsynephrine hydrochloride (Formula shown in Example 12, above) is used here as a non-limiting example, even though another Synephrine Derivative may be used in its place in this Example:

TABLE 1

| Components | Amount (mg) |
|---|---|
| Isopropylnorsyneohrine hydrochloride | 15 |
| Starch | 80 |
| Microcrystal cellulose | 100 |
| Magnesium stearate | 15 |
| Lactose | 40 |
| TOTAL | 250 |

### Example 23

### Non-Limiting Example of a Capsule Formulation

The Synephrine Derivatives may be formulated in capsule form as shown below. For the sake of convenience, the known compound isopropylnorsynephrine hydrochloride (Formula shown in Example 12, above) is used here as a non-limiting example, even though another Synephrine Derivative may be used in its place in this Example.

Isopropylnorsynephrine hydrochloride, corn starch, and magnesium stearate are mixed together at the weight ratio of 1:40:4. The resulting mixture is filled into blue size 0 snap-fit capsules The capsule's filled weight is about 300 mg.

### Example 24

### Non-Limiting Example of a Weight Loss Test with Volunteer Subjects

The efficacy of the Synephrine Derivatives may be tested according to the following regimen.

A test group of 8 human volunteer subjects (4 male and 4 female, age from 23 to 41, Body Mass Index ranging from 26 to 32) take two of the above capsules of Example 23, at a dose of 3 times daily for four weeks, which corresponds to an intake of 13.5 mg total active ingredient per serving, or 40 mg per day, without deliberate restriction of food intake. Weights at 0 day, 7 days and 14 days are determined.

If a statistically significant weight loss is found (P<0.05) during two weeks taking the capsule, it would tend to indicate that the tested Synephrine Derivative has an effect on weight loss. Then, at the discretion of the experimenter, a larger clinical studies with more subjects may be undertaken.

### Example 25

### Decrease in Body Fat

Activity of the Synephrine Derivatives for decrease in body fat can be determined according to the following procedure. C57BL/6J ob/ob mice (male, 30-40 g body weight, Jackson Lab, Bar Harbor, Me.) are housed five mice per cage in an environmentally controlled room with food (pelleted rodent chow) and water available ad libitum. The Synephrine Derivative or vehicle (e.g. 0.5% w/v methyl cellulose/distilled water, water, or other suitable vehicle) is dosed once or twice daily for three weeks (0.01-20 mg/Kg, n=15 per group) by oral gavage. Body weight of each mouse is measured daily and food intake per cage determined by weighing the amount of food left in the trough. At the end of the study, twenty-four hours after giving the final dose of compound, the mice are weighed and then sacrificed by cervical dislocation. The epididymal fat pads from each mouse are excised and weighed. The fat versus body weight ratio is determined for each mouse using the absolute body weights and the fat pad weights. A reduction in fat pad weight is indicative of a reduction in total body fat.

Although the foregoing invention has been described in some detail by way of illustration and example for purposes of clarity and understanding, it will be obvious that various modifications and changes which are within the skill of those skilled in the art are considered to fall within the scope of the appended claims. Future technological advancements which allows for obvious changes in the basic invention herein are also within the claims.

1. A method for synthesizing a compound of Formula III, comprising the step of reacting a phenol of Formula II with a haloacetyl halide, in the presence of a Lewis acid catalyst, to produce a compound of Formula III, the reaction step is graphically presented below: 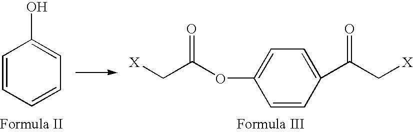
wherein x is a halo substituent selected from the group consisting of chloro, bromo, iodo, and fluoro:

2. The method for synthesizing a compound of Formula I, said method comprising the method of claim 1 as its step 1, followed by a step 2, wherein the compound of Formula III is reacted with an amine of Formula IV to produce a compound of Formula V; and a step 3, wherein the compound of Formula V is reduced to produce the compound of Formula I; wherein the method is graphically presented below: 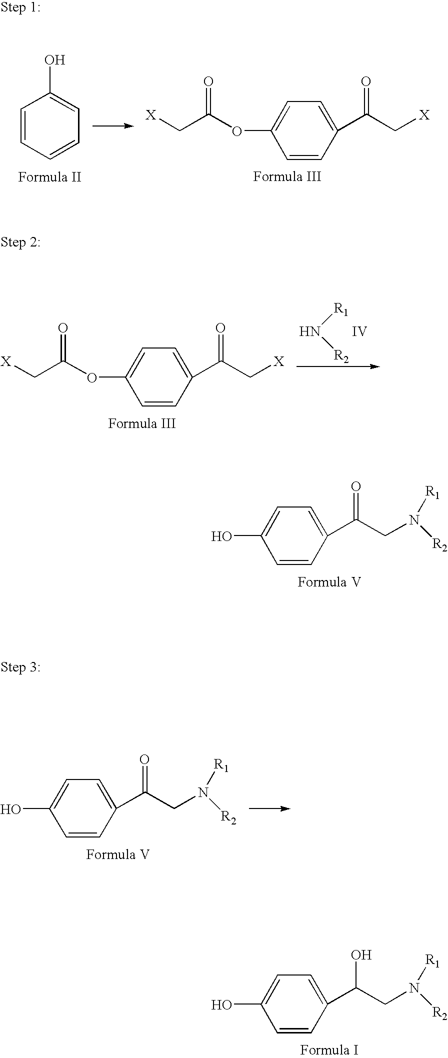
wherein:
X is as defined in claim 1,
R₁ and R₂ can be the same or different,
R₁ and R₂ can be separate or can be bonded together,
If R₁ and R₂ are separate, R₁ and R₂ are selected from the group consisting of:
(a) a hydrogen;
(b) a C₁ to C₆ alkyl group, the alkyl may be independently unsubstituted or substituted with 1 to 2 substituents independently selected from item (k), below;
(c) a C₁ to C₆ alkyl group which is attached to a phenyl which may be independently unsubstituted or substituted with 1 to 5 substituents independently selected from item (k), below;
(d) a C₁ to C₆ alkyl group which is attached to a 5- or 6-membered aromatic heterocyclic ring, the heterocyclic ring has 1 or 2 heteroatoms independently selected from the group consisting of O, S, and N; the 5- or 6-membered aromatic heterocyclic ring may be independently unsubstituted or substituted with 1 to 4 substituents; the substituents are independently selected from item (k), below;
(e) a C₁ to C₆ alkyl group which is attached to a 5- or 6-membered non-aromatic heterocyclic ring, the heterocyclic ring has 1 or 2 heteroatoms independently selected from the group consisting of O, S, and N; the 5- or 6-membered non-aromatic heterocyclic ring may be independently unsubstituted or substituted with 1 to 4 substituents independently selected from item (k), below;
(f) a C₃ to C₆ cycloalkyl, the cycloalkyl may be independently unsubstituted or substituted with 1 to 2 substituents independently selected from item (k), below;
(g) a C₃ to C₆ cycloalkyl group which is fused to a phenyl, the phenyl may be independently unsubstituted or substituted with 1 to 5 substituents independently selected from item (k), below;
(h) a phenyl which may be independently unsubstituted or substituted with 1 to 5 substituents independently selected from item (k), below;
(i) a 5- or 6-membered aromatic heterocyclic ring having 1 or 2 heteroatoms independently selected from the group consisting of O, S, and N; the 5- or 6-membered aromatic heterocyclic ring may be independently unsubstituted or substituted with 1 to 4 substituents independently selected from item (k), below;
(j) a 5- or 6-membered non-aromatic heterocyclic ring having 1 or 2 heteroatoms independently selected from the group consisting of O, S, and N; the 5- or 6-membered non-aromatic heterocyclic ring may be independently unsubstituted or substituted with 1 to 4 substituents independently selected from item (k), below;
(k) In items (b) to (j), above, where there are substituents, the substituents are independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine;
In the case where R₁ and R₂ are bonded together, they are bonded together to include the N of Formula I to form a 5- or 6-membered heterocyclic ring, this N is counted as one heteroatom, an optional heteroatom may be included in the heterocyclic ring; the additional heteroatom is selected from the group consisting of O, S, and N; the heterocyclic ring may be independently unsubstituted or substituted with 1 to 4 substituents independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine.

3. The method of claim 2, wherein an intermediate of Formula VI is formed in step 2, as graphically presented below: 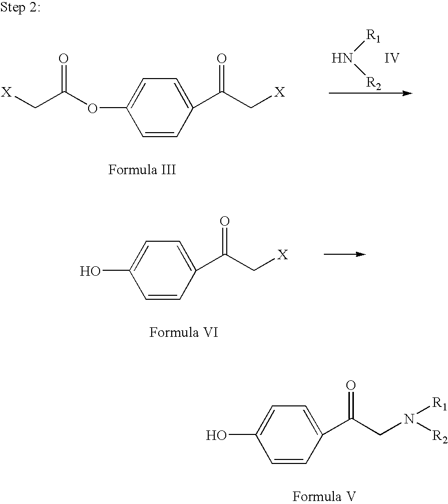
Wherein X is as defined in claim 1.

4. The method of claim 2, wherein:
(a) in step 1, the haloacetyl halide is chloroacetyl chloride and the reaction takes place under Friedel-Crafts reaction conditions; and
(b) in step 3, the compound of Formula V is reduced to Formula I according to the method selected from the group consisting of: reduction with hydrogen in the presence of a catalyst, and reduction with a reducing agent.

5. The method of claim 2, wherein the compound of Formula I is selected from the group consisting of:
(a) 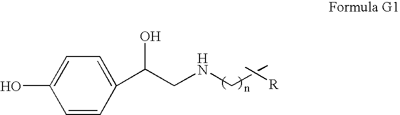
wherein n is between 0 to 5;
(b) 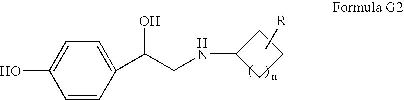
wherein n is between 0 to 3; 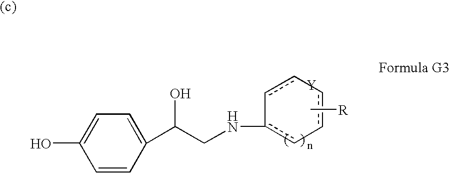
wherein n is between 0 to 1; 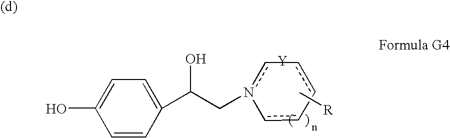
wherein n is between 0 to 1; 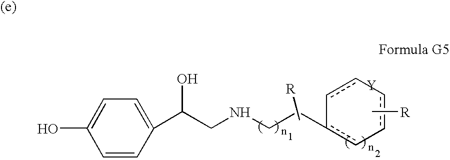
wherein n₁ is between 0 to 5; n₂ is between 0 to 1; and
wherein in Formulae G1 to G5, R is selected from the group consisting of: H, hydroxy, halo, cyano, nitro, amino, phenyl, benzyl, CF₃, C₁ to C₆ alkyl, C₁ to C₆ alkoxy, C₁ to C₆ alkylol, and C₁ to C₆ alkylamine; and “Y” is selected from the group consisting of: CH, CH₂, N, NH, O, and S.

6. The method of claim 2, wherein the compound of Formula I is selected from the group consisting of:

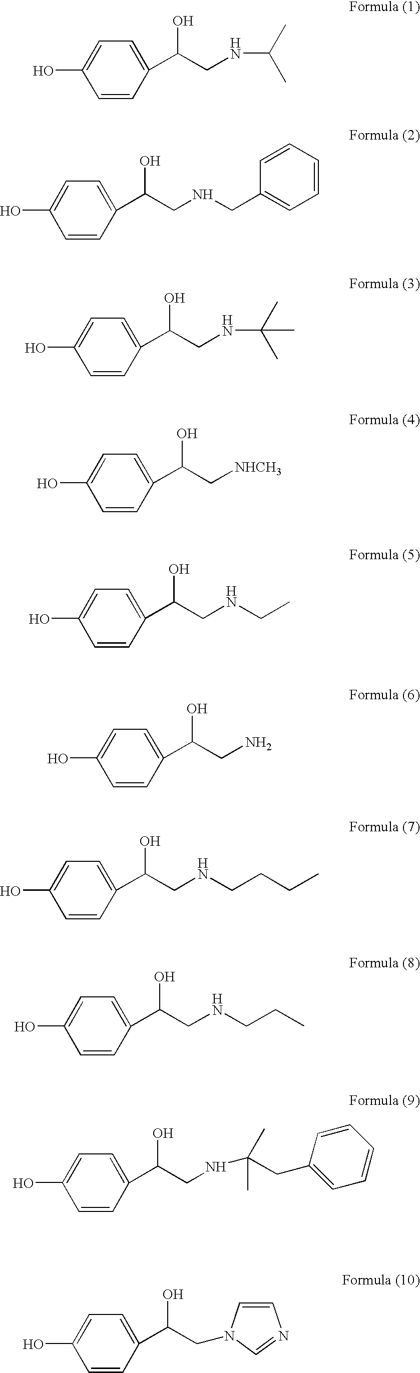

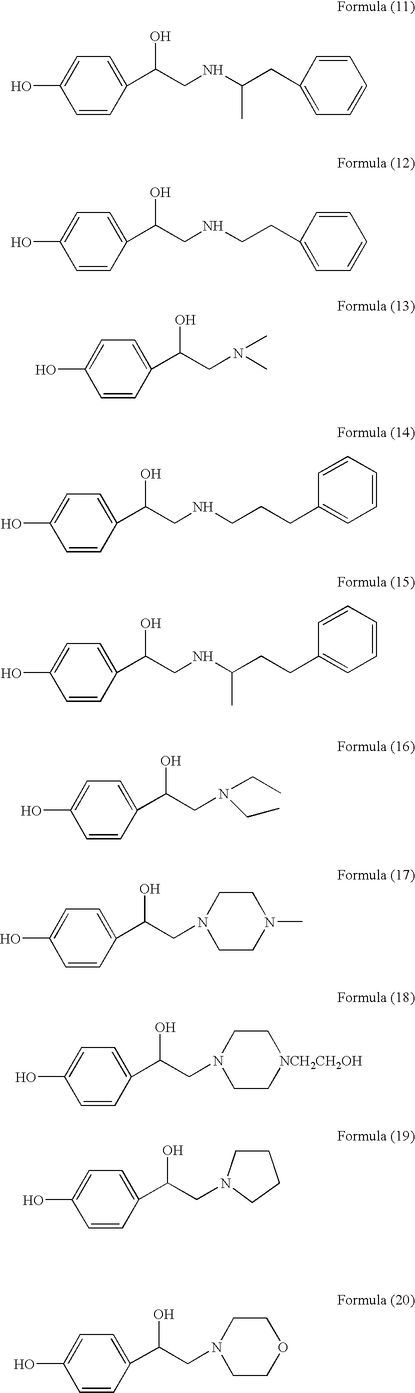

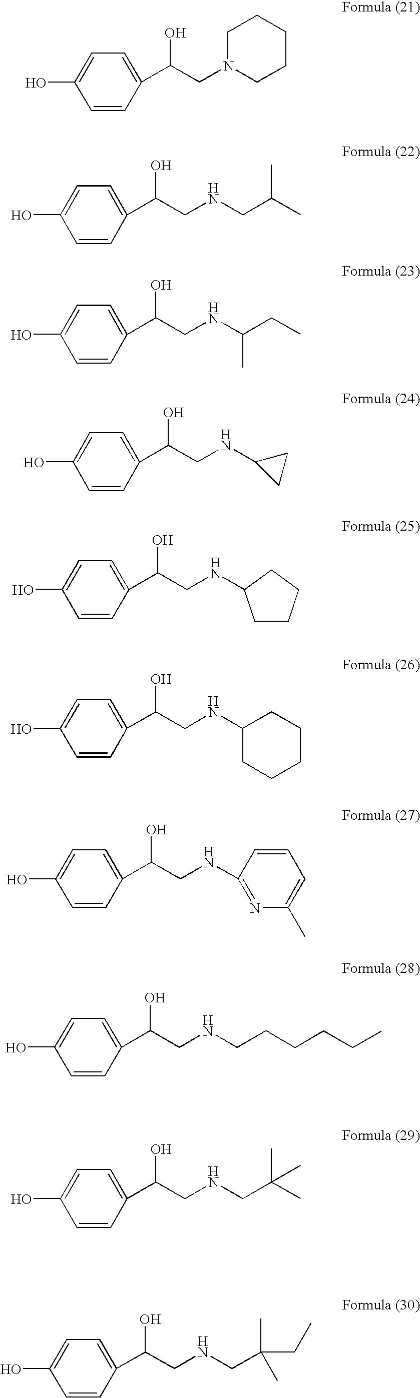

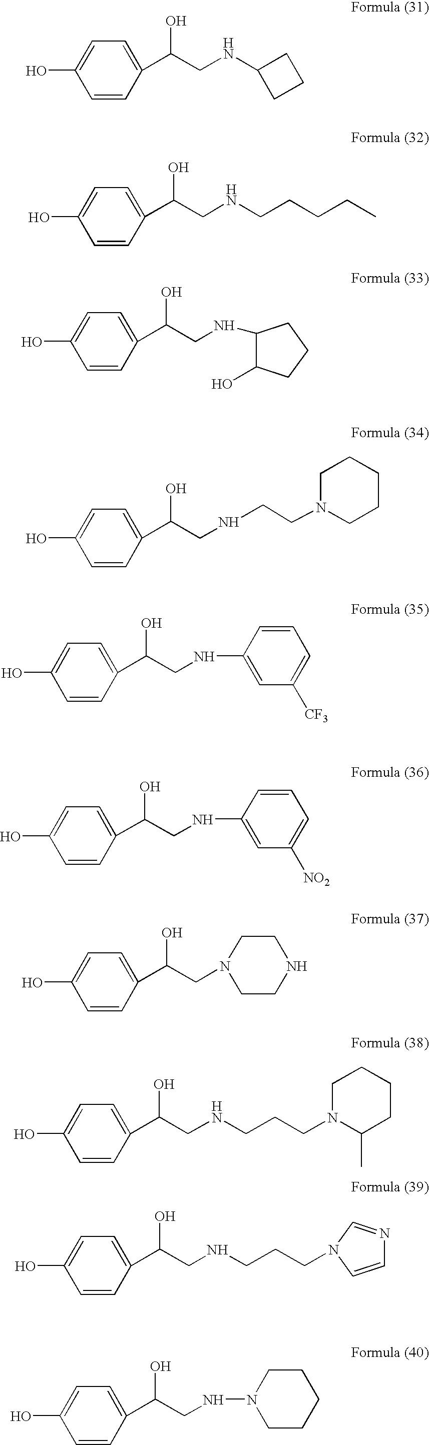

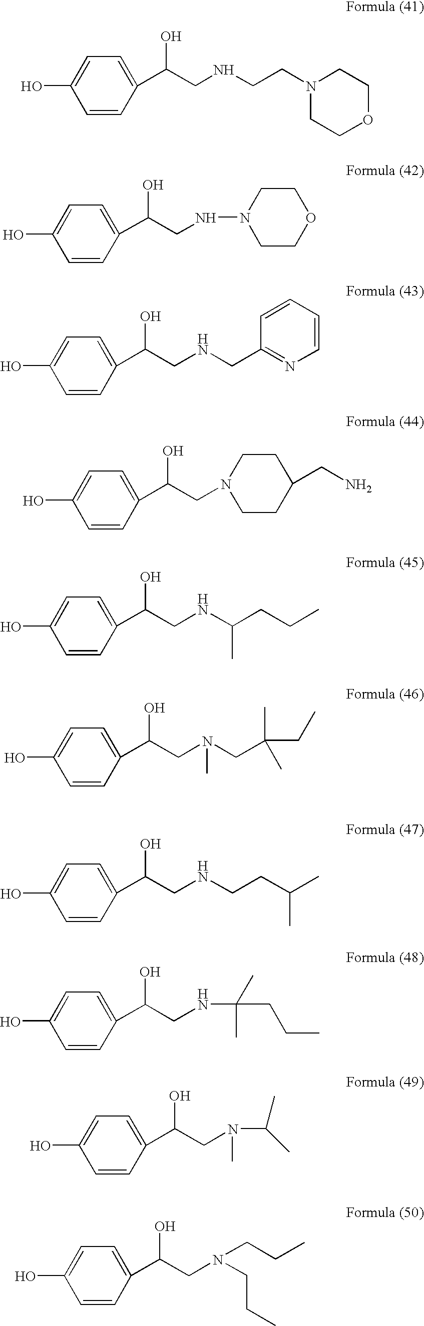

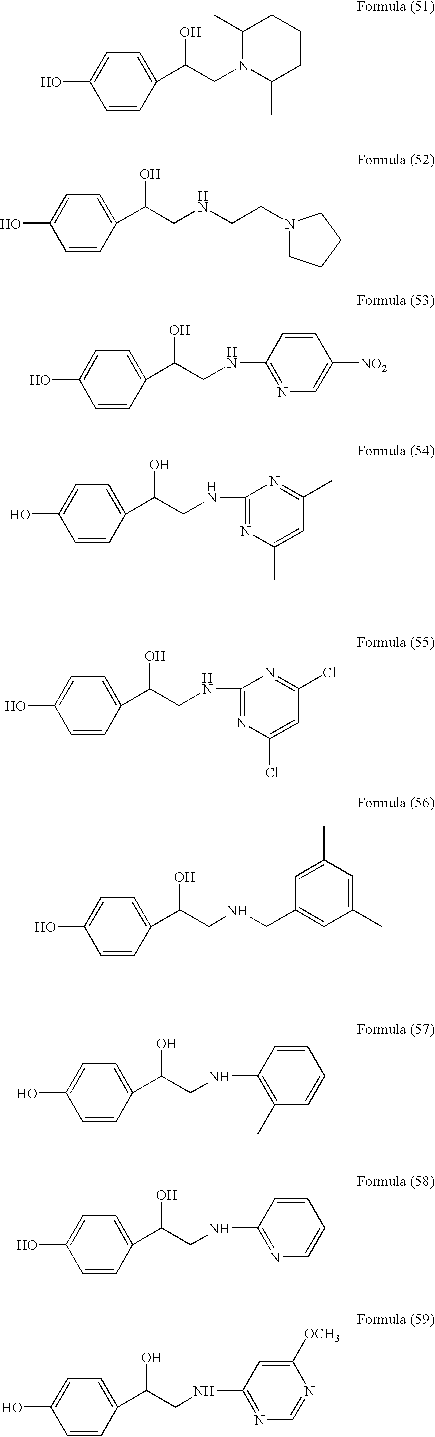

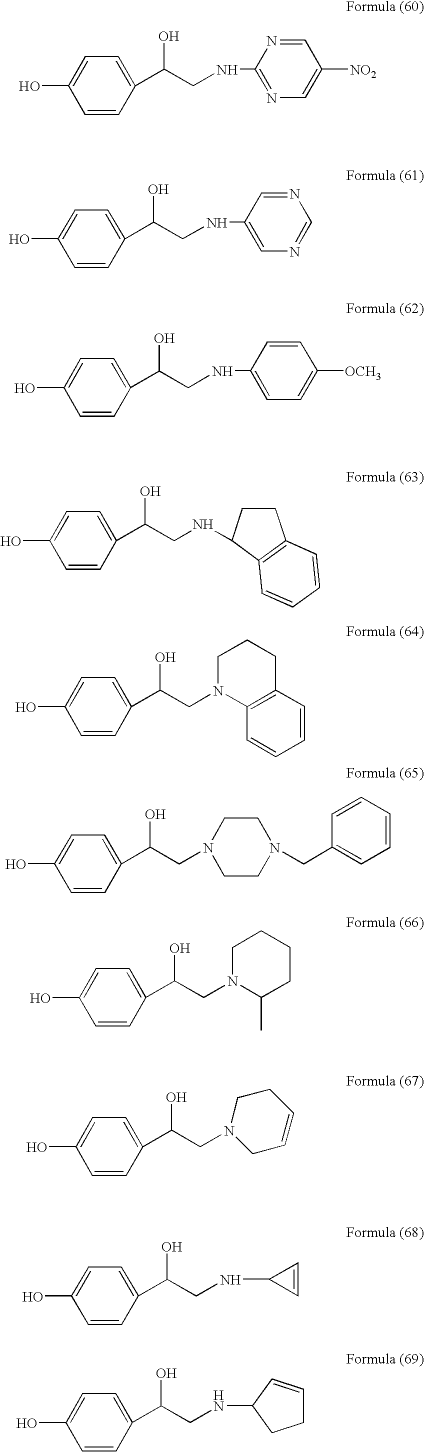

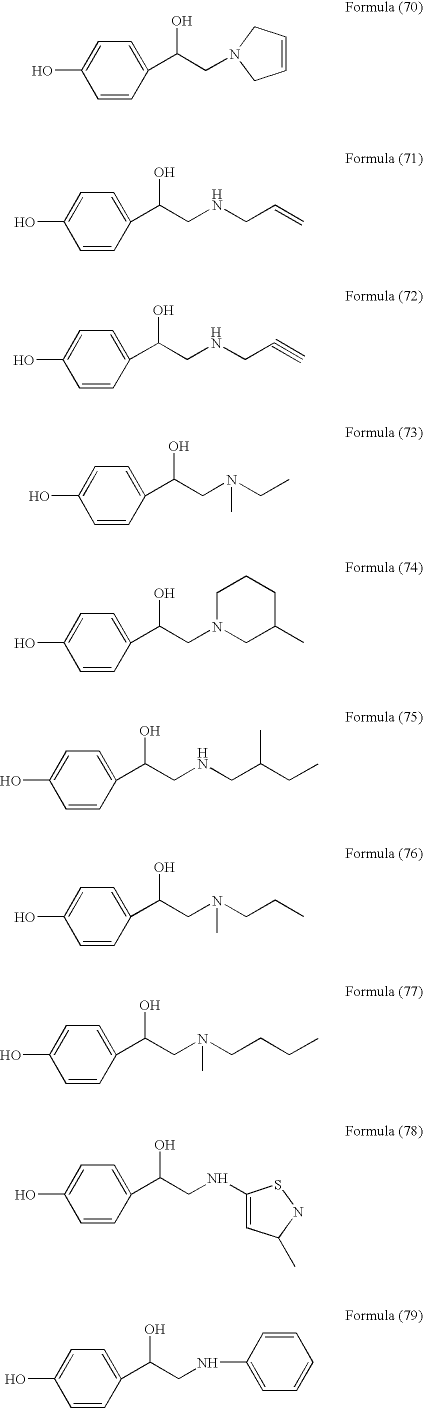

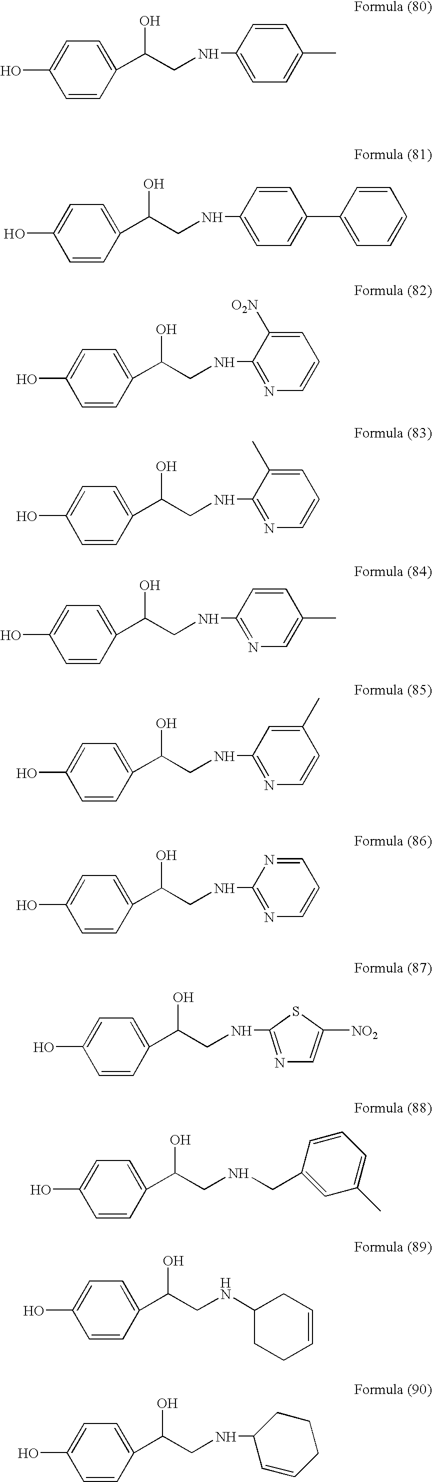

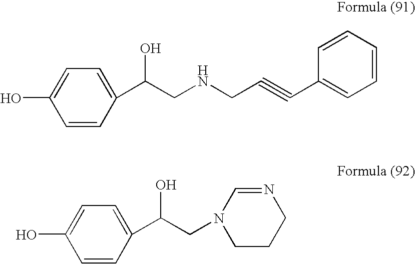

7. The method of claim 2, further comprising the step of reacting the compound of Formula I with an acid to produce a corresponding salt of the compound of Formula I.

8. The method of claim 5, further comprising the step of reacting the compound of Formula I with an acid to produce a corresponding salt of the compound of Formula I.

9. The method of claim 6, further comprising the step of reacting the compound of Formula I with an acid to produce a corresponding salt of the compound of Formula I.

10. A compound represented by a chemical formula selected from the group consisting of the following formulae and their corresponding salts:

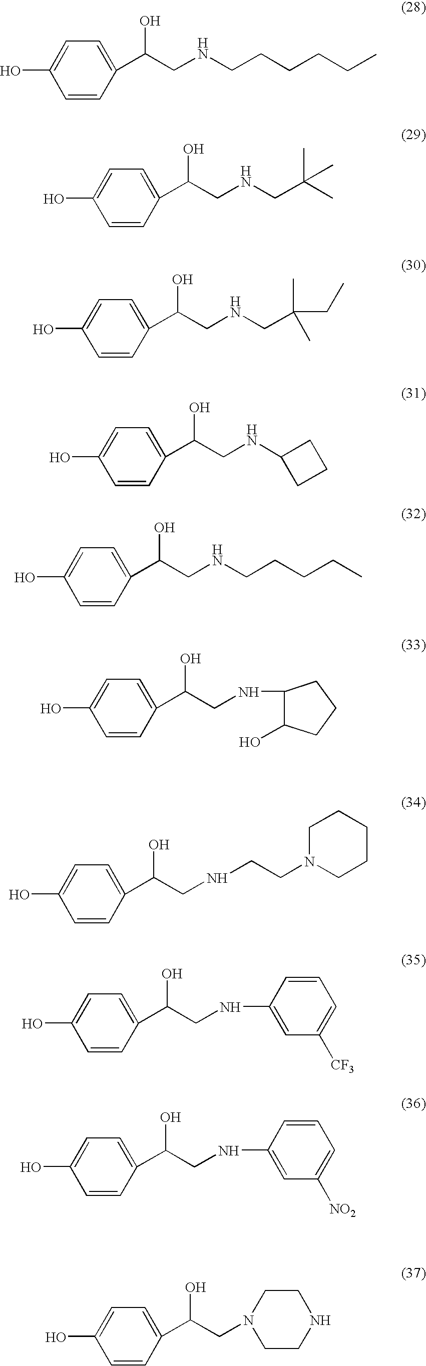

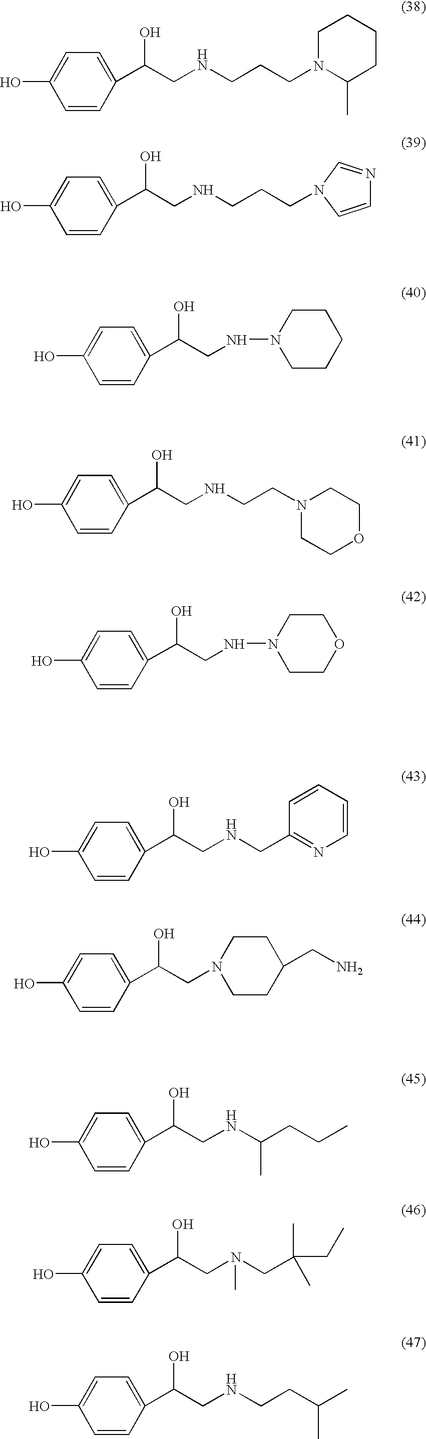

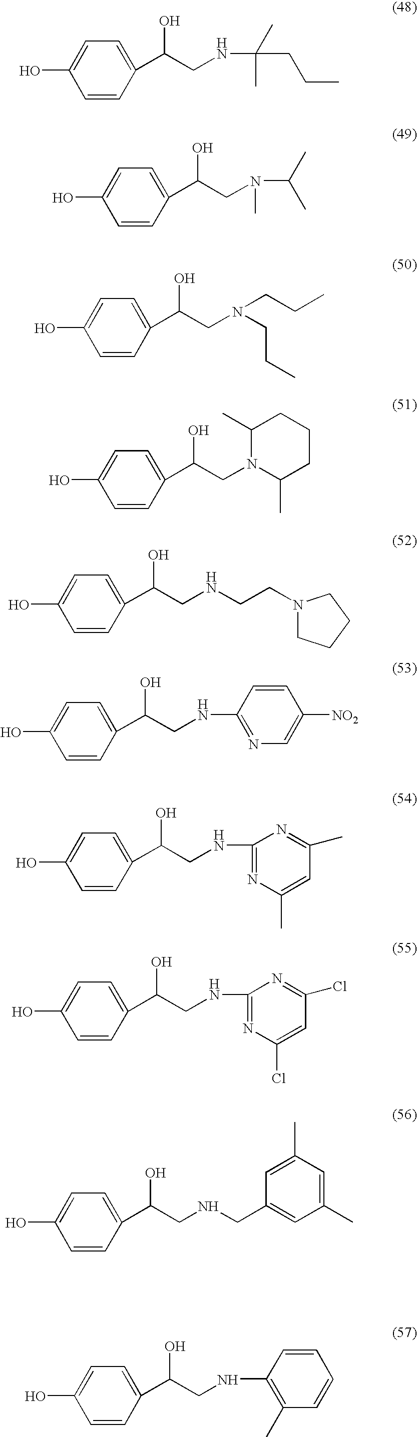

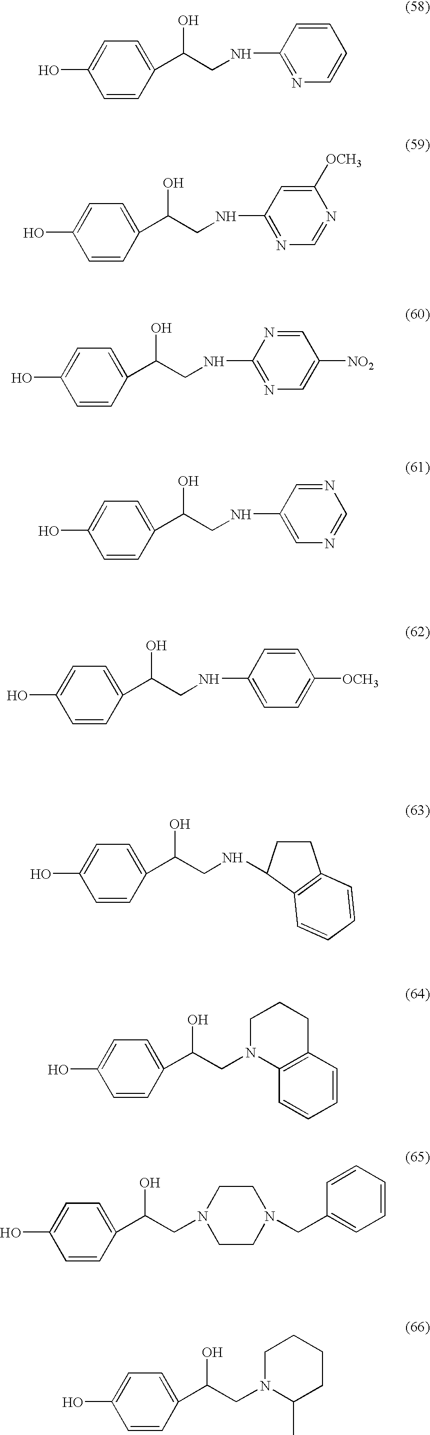

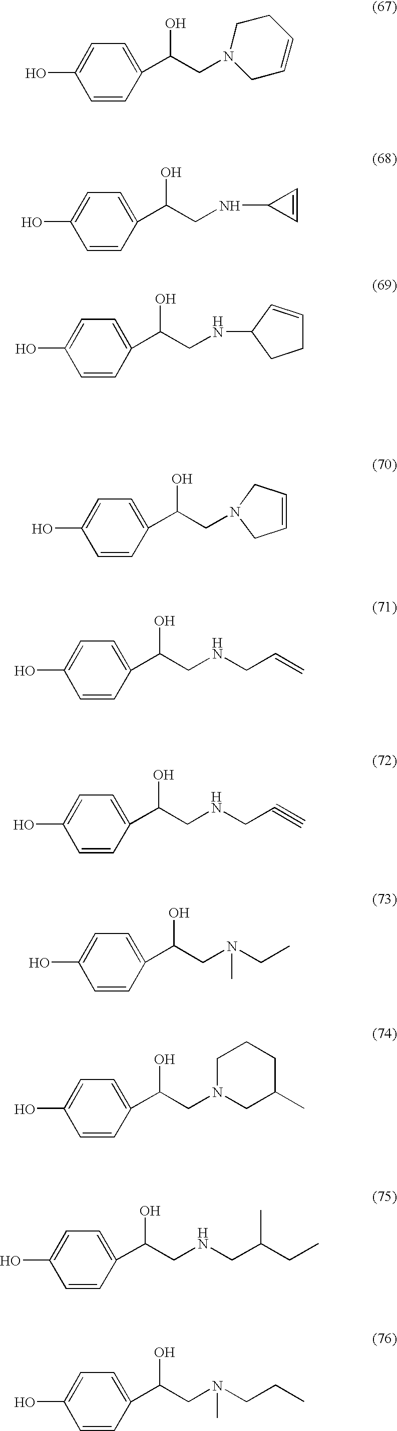

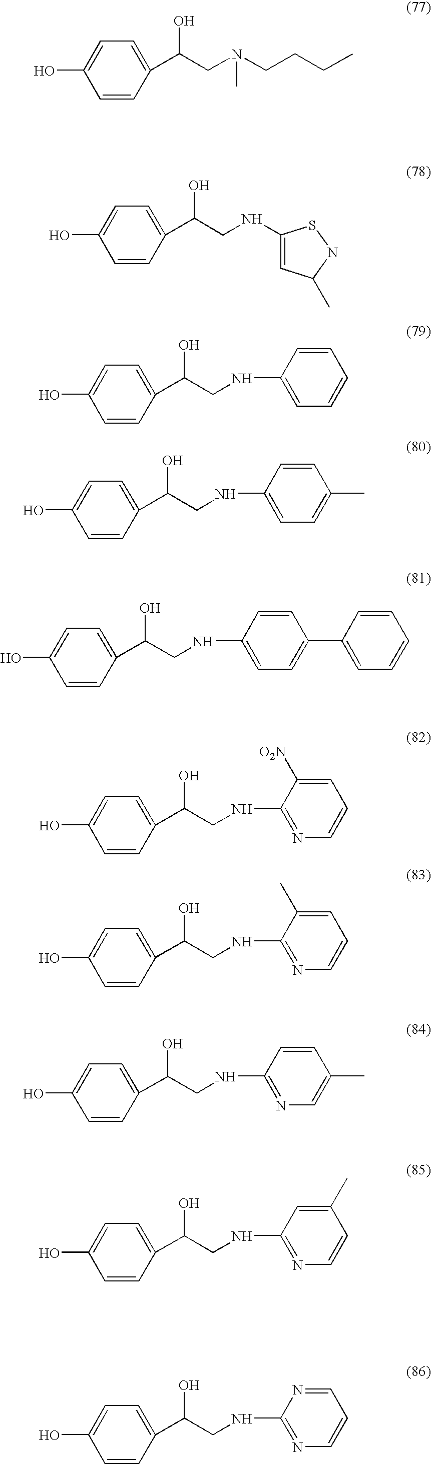

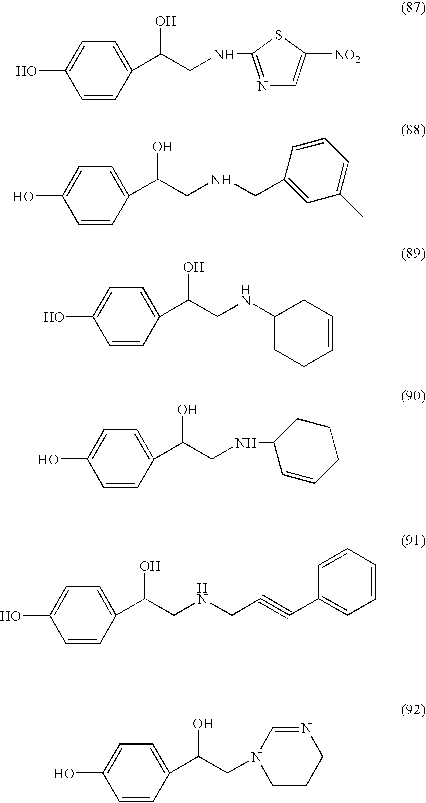

11. A method for treating a disease, condition, or disorder in an animal which can be modulated by a compound with β₃ adrenergic activity, comprising the step of administering to the animal or contacting the animal with a compound of Formula I, the salt thereof, a prodrug of said compound or said salt, a solvate or hydrate of said compound, said salt, or said prodrug; wherein Formula I is represented below: 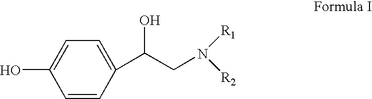
wherein:
R₁ and R₂ can be the same or different,
R₁ and R₂ can be separate or can be bonded together,
If R₁ and R₂ are separate, R₁ and R₂ are selected from the group consisting of:
(a) a hydrogen;
(b) a C₁ to C₆ alkyl group, the alkyl may be independently unsubstituted or substituted with 1 to 2 substituents independently selected from item (k), below;
(c) a C₁ to C₆ alkyl group which is attached to a phenyl which may be independently unsubstituted or substituted with 1 to 5 substituents independently selected from item (k), below;
(d) a C₁ to C₆ alkyl group which is attached to a 5- or 6-membered aromatic heterocyclic ring, the heterocyclic ring has 1 or 2 heteroatoms independently selected from the group consisting of O, S, and N; the 5- or 6-membered aromatic heterocyclic ring may be independently unsubstituted or substituted with 1 to 4 substituents; the substituents are independently selected from item (k), below;
(e) a C₁ to C₆ alkyl group which is attached to a 5- or 6-membered non-aromatic heterocyclic ring, the heterocyclic ring has 1 or 2 heteroatoms independently selected from the group consisting of O, S, and N; the 5- or 6-membered non-aromatic heterocyclic ring may be independently unsubstituted or substituted with 1 to 4 substituents independently selected from item (k), below;
(f) a C₃ to C₆ cycloalkyl, the cycloalkyl may be independently unsubstituted or substituted with 1 to 2 substituents independently selected from item (k), below;
(g) a C₃ to C₆ cycloalkyl group which is fused to a phenyl, the phenyl may be independently unsubstituted or substituted with 1 to 5 substituents independently selected from item (k), below;
(h) a phenyl which may be independently unsubstituted or substituted with 1 to 5 substituents independently selected from item (k), below;
(i) a 5- or 6-membered aromatic heterocyclic ring having 1 or 2 heteroatoms independently selected from the group consisting of O, S, and N; the 5- or 6-membered aromatic heterocyclic ring may be independently unsubstituted or substituted with 1 to 4 substituents independently selected from item (k), below;
(j) a 5- or 6-membered non-aromatic heterocyclic ring having 1 or 2 heteroatoms independently selected from the group consisting of O, S, and N; the 5- or 6-membered non-aromatic heterocyclic ring may be independently unsubstituted or substituted with 1 to 4 substituents independently selected from item (k), below;
(k) In items (b) to (j), above, where there are substituents, the substituents are independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine;
In the case where R₁ and R₂ are bonded together, they are bonded together to include the N of Formula I to form a 5- or 6-membered heterocyclic ring, this N is counted as one heteroatom, an optional heteroatom may be included in the heterocyclic ring; the additional heteroatom is selected from the group consisting of O, S, and N; the heterocyclic ring may be independently unsubstituted or substituted with 1 to 4 substituents independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine; and Formula I excludes octopamine and synephrine.

12. The method of claim 11, wherein the compound of Formula I is selected from the group consisting of: 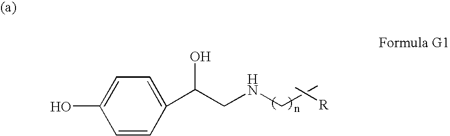
wherein n is between 0 to 5; 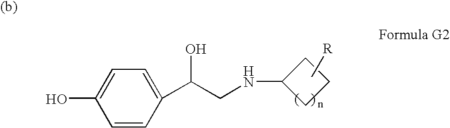
wherein n is between 0 to 3; 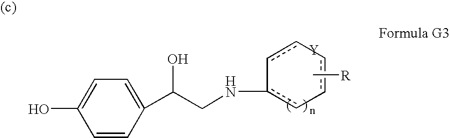
wherein n is between 0 to 1; 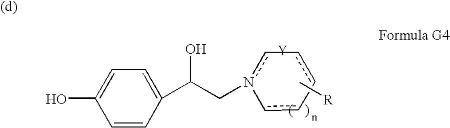
wherein n is between 0 to 1; 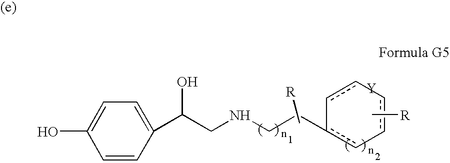
wherein n₁ is between 0 to 5; n₂ is between 0 to 1; and
wherein in Formulae G1 to G5, R is selected from the group consisting of: H, hydroxy, halo, cyano, nitro, amino, phenyl, benzyl, CF₃, C₁ to C₆ alkyl, C₁ to C₆ alkoxy, C₁ to C₆ alkylol, and C₁ to C₆ alkylamine; and “Y” is selected from the group consisting of: CH, CH₂, N, NH, O, and S.

13. The method of claim 11, wherein the compound of Formula I is selected from the group consisting of:

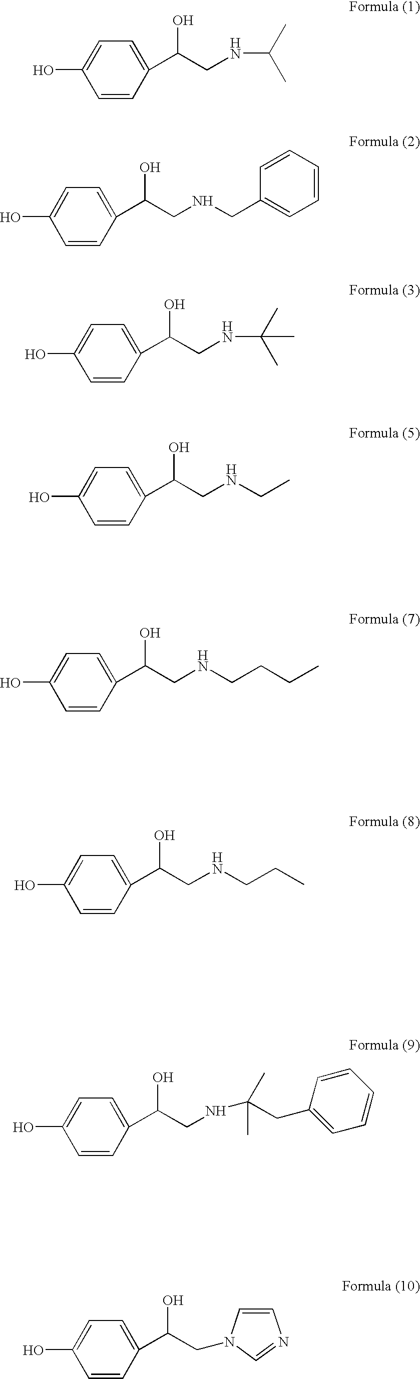

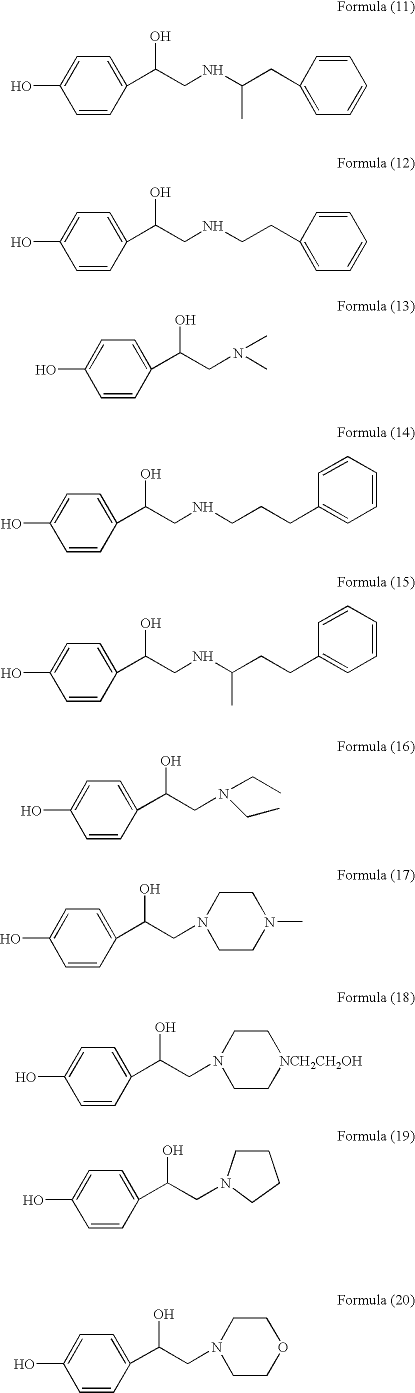

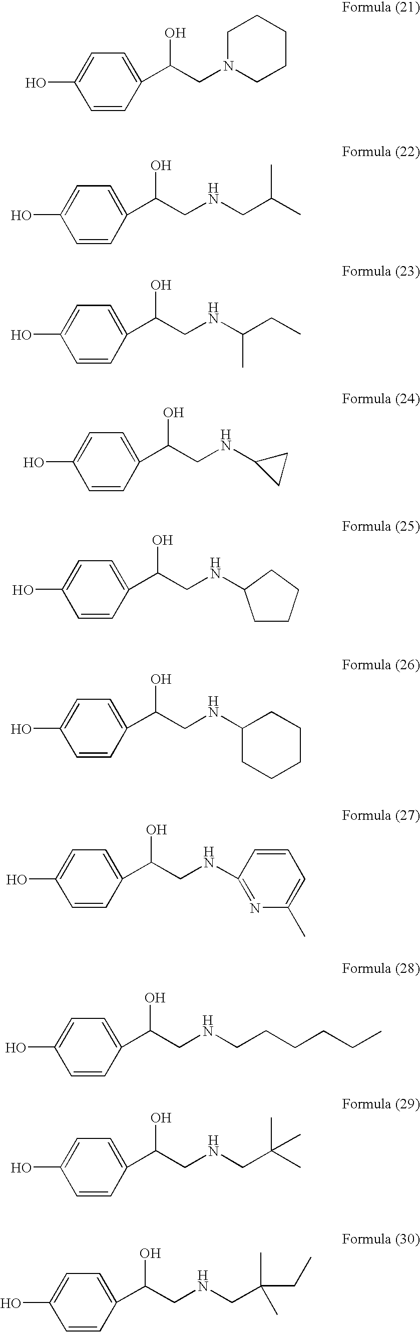

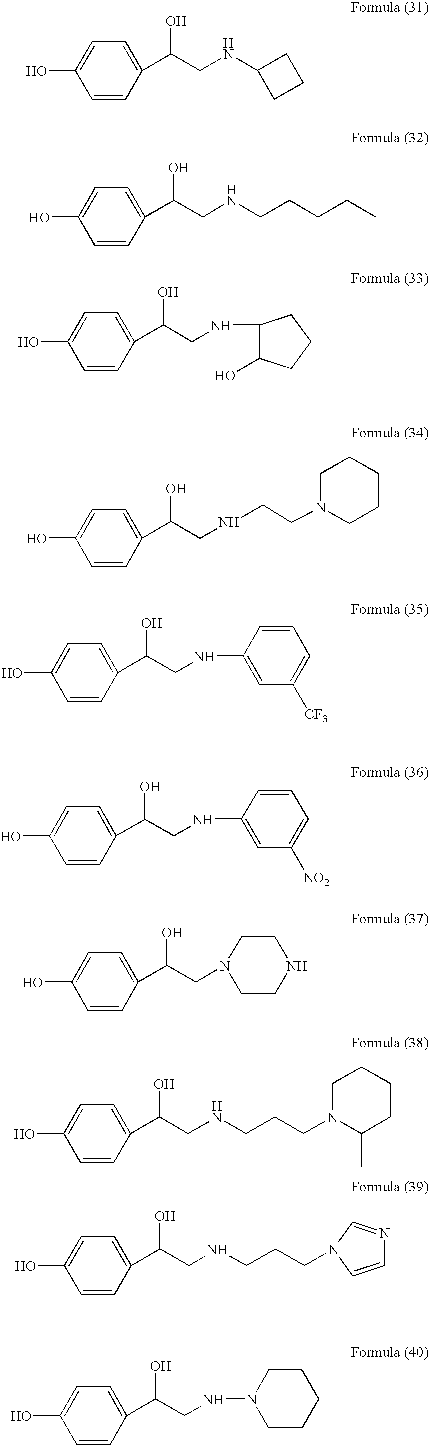

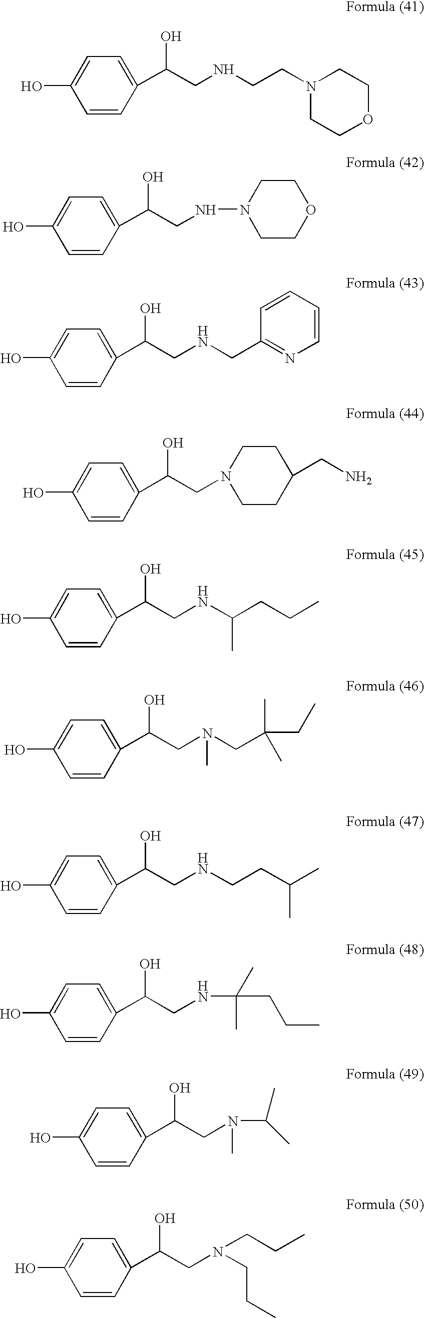

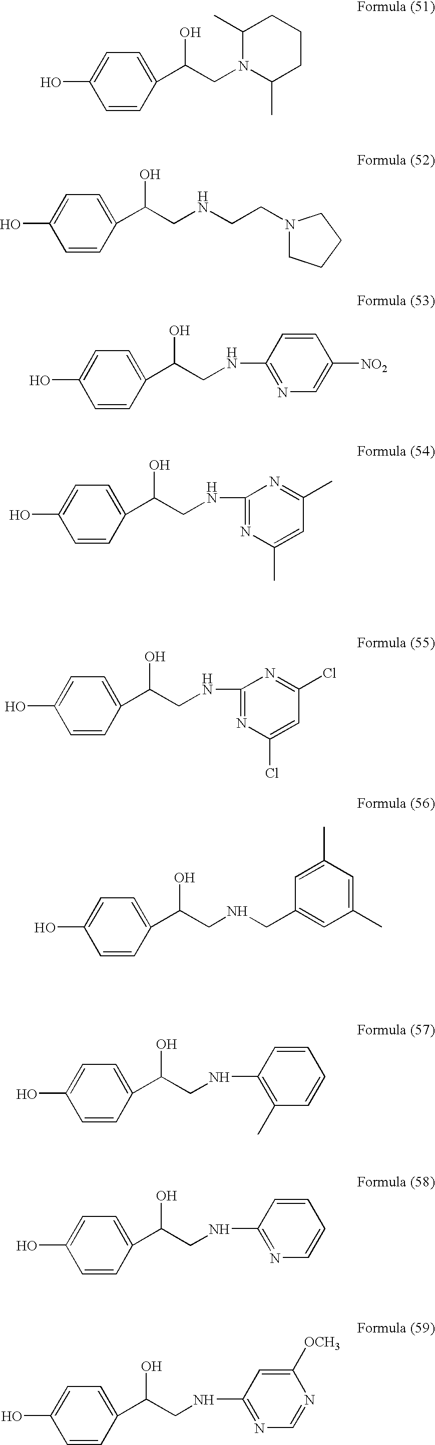

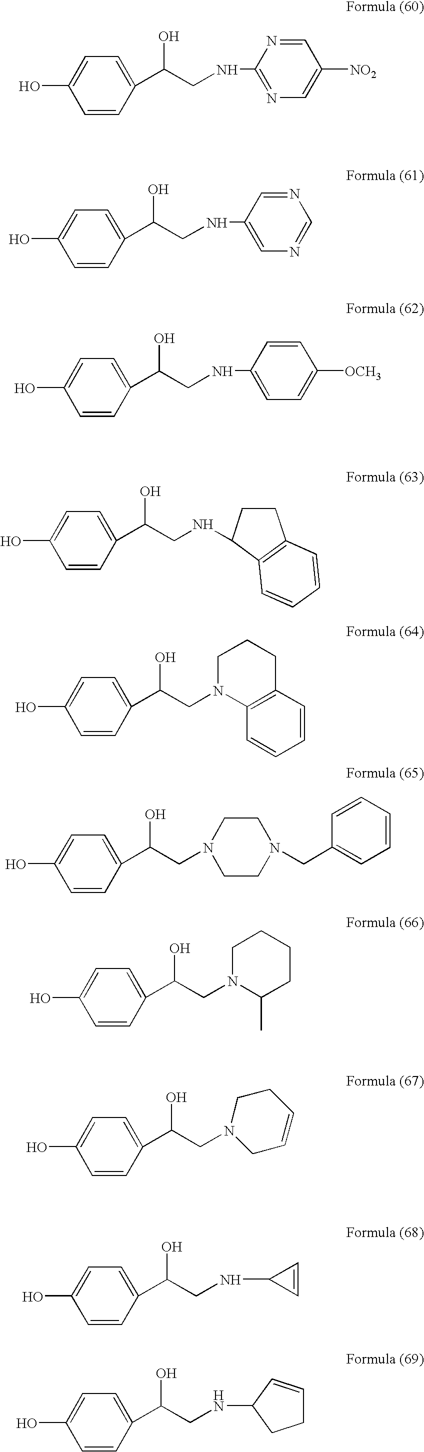

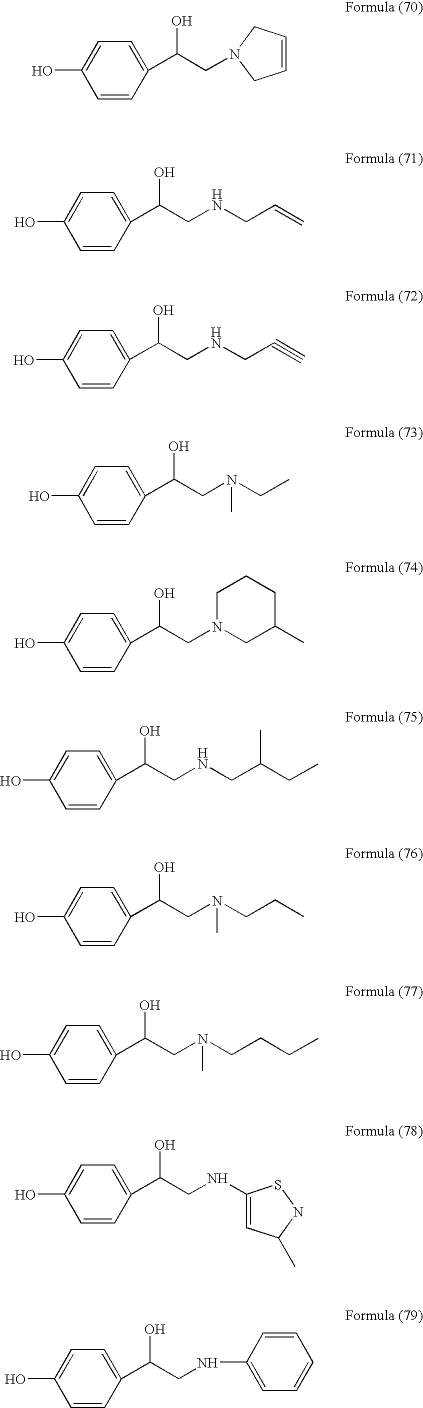

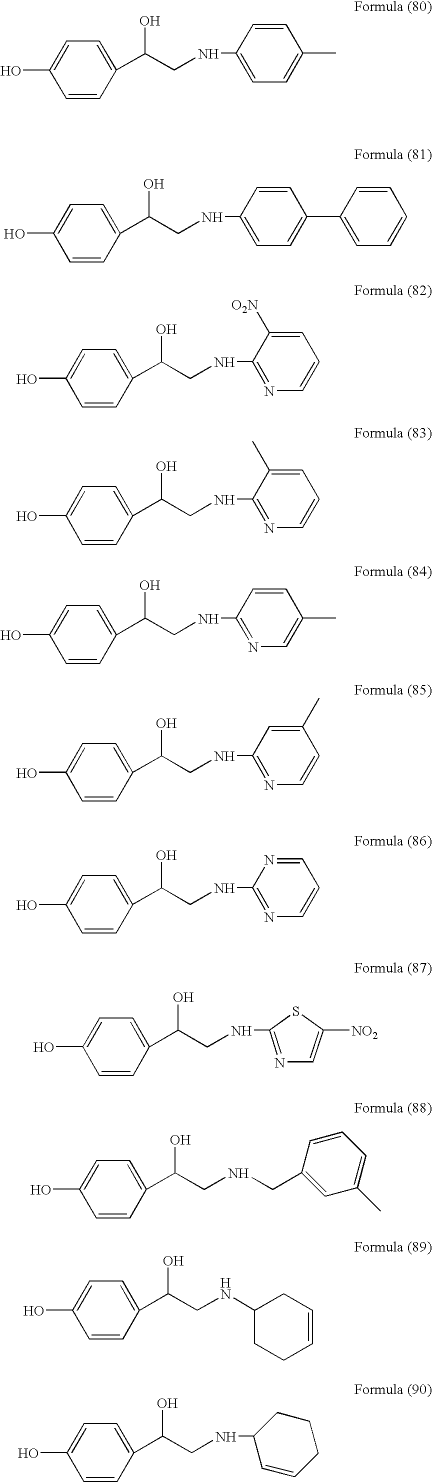

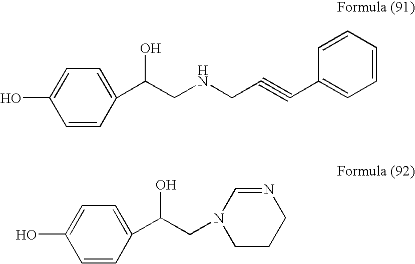

14. The method of claim 11, wherein the compound of Formula I is selected from the group consisting of:

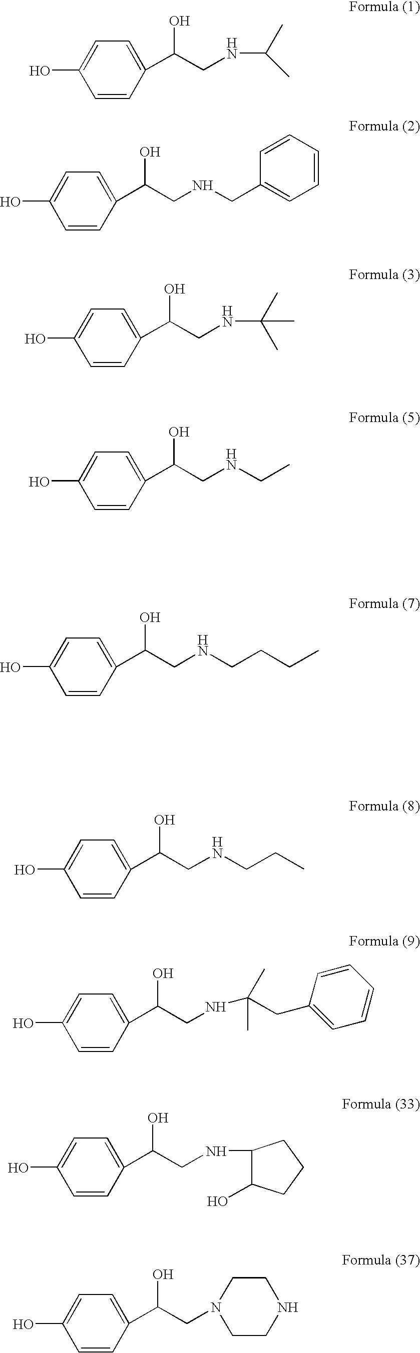

15. The method of claim 11, wherein the disease, condition or disorder is obesity, the animal is a mammal, and the compound or its salt stimulates body fat breakdown or weight loss in the mammal.

16. The method of claim 12, wherein the disease, condition or disorder is obesity, the animal is a mammal, and the compound or its salt stimulates body fat breakdown or weight loss in the mammal.

17. A pharmaceutical composition comprising: (1) a therapeutically effective amount of a compound of Formula I, the salt thereof, a prodrug of said compound or said salt, a solvate or hydrate of said compound, said salt, or said prodrug; and (2) a pharmaceutically acceptable carrier; wherein said therapeutically effective amount is effective for stimulating body fat breakdown or weight loss in a mammal, and wherein Formula I is represented below: 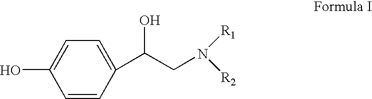
Wherein:
R₁ and R₂ can be the same or different,
R₁ and R₂ can be separate or can be bonded together,
If R₁ and R₂ are separate, R₁ and R₂ are selected from the group consisting of:
(a) a hydrogen;
(b) a C₁ to C₆ alkyl group, the alkyl may be independently unsubstituted or substituted with 1 to 2 substituents independently selected from item (k), below;
(c) a C₁ to C₆ alkyl group which is attached to a phenyl which may be independently unsubstituted or substituted with 1 to 5 substituents independently selected from item (k), below;
(d) a C₁ to C₆ alkyl group which is attached to a 5- or 6-membered aromatic heterocyclic ring, the heterocyclic ring has 1 or 2 heteroatoms independently selected from the group consisting of O, S, and N; the 5- or 6-membered aromatic heterocyclic ring may be independently unsubstituted or substituted with 1 to 4 substituents; the substituents are independently selected from item (k), below;
(e) a C₁ to C₆ alkyl group which is attached to a 5- or 6-membered non-aromatic heterocyclic ring, the heterocyclic ring has 1 or 2 heteroatoms independently selected from the group consisting of O, S, and N; the 5- or 6-membered non-aromatic heterocyclic ring may be independently unsubstituted or substituted with 1 to 4 substituents independently selected from item (k), below;
(f) a C₃ to C₆ cycloalkyl, the cycloalkyl may be independently unsubstituted or substituted with 1 to 2 substituents independently selected from item (k), below;
(g) a C₃ to C₆ cycloalkyl group which is fused to a phenyl, the phenyl may be independently unsubstituted or substituted with 1 to 5 substituents independently selected from item (k), below;
(h) a phenyl which may be independently unsubstituted or substituted with 1 to 5 substituents independently selected from item (k), below;
(i) a 5- or 6-membered aromatic heterocyclic ring having 1 or 2 heteroatoms independently selected from the group consisting of O, S, and N; the 5- or 6-membered aromatic heterocyclic ring may be independently unsubstituted or substituted with 1 to 4 substituents independently selected from item (k), below;
(j) a 5- or 6-membered non-aromatic heterocyclic ring having 1 or 2 heteroatoms independently selected from the group consisting of O, S, and N; the 5- or 6-membered non-aromatic heterocyclic ring may be independently unsubstituted or substituted with 1 to 4 substituents independently selected from item (k), below;
(k) In items (b) to (j), above, where there are substituents, the substituents are independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine;
In the case where R₁ and R₂ are bonded together, they are bonded together to include the N of Formula I to form a 5- or 6-membered heterocyclic ring, this N is counted as one heteroatom, an optional heteroatom may be included in the heterocyclic ring; the additional heteroatom is selected from the group consisting of O, S, and N; the heterocyclic ring may be independently unsubstituted or substituted with 1 to 4 substituents independently selected from the group consisting of: hydroxy, halogen, cyano, nitro, amino, phenyl, benzyl, CF₃, C₁ to C₆ alkyl, C₁ to C₆ alkoxy, C₁ to C₆ alkylol, and C₁ to C₆ alkylamine; and Formula I excludes octopamine and synephrine.

18. The pharmaceutical composition of claim 17, wherein the compound of Formula I is selected from the group consisting of: 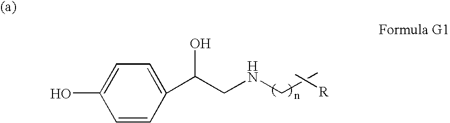
wherein n is between 0 to 5; 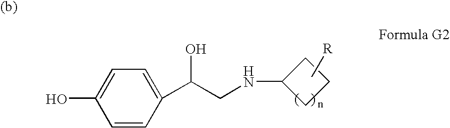
wherein n is between 0 to 3; 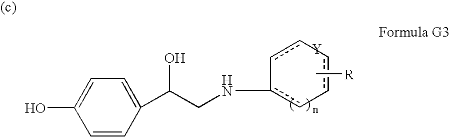
wherein n is between 0 to 1; 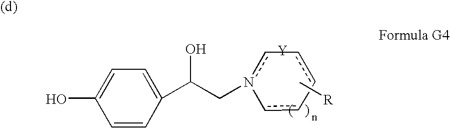
wherein n is between 0 to 1; 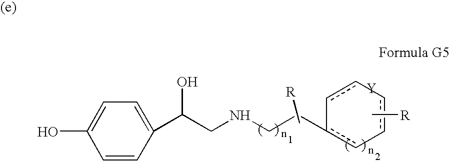
wherein n, is between 0 to 5; n₂ is between 0 to 1; and
wherein in Formulae G1 to G5, R is selected from the group consisting of: H, hydroxy, halo, cyano, nitro, amino, phenyl, benzyl, CF₃, C₁ to C₆ alkyl, C₁ to C₆ alkoxy, C₁ to C₆ alkylol, and C₁ to C₆ alkylamine; and “Y” is selected from the group consisting of: CH, CH₂, N, NH, O, and S.

19. The pharmaceutical composition of claim 17, wherein the compound of Formula I is selected from the group consisting of:

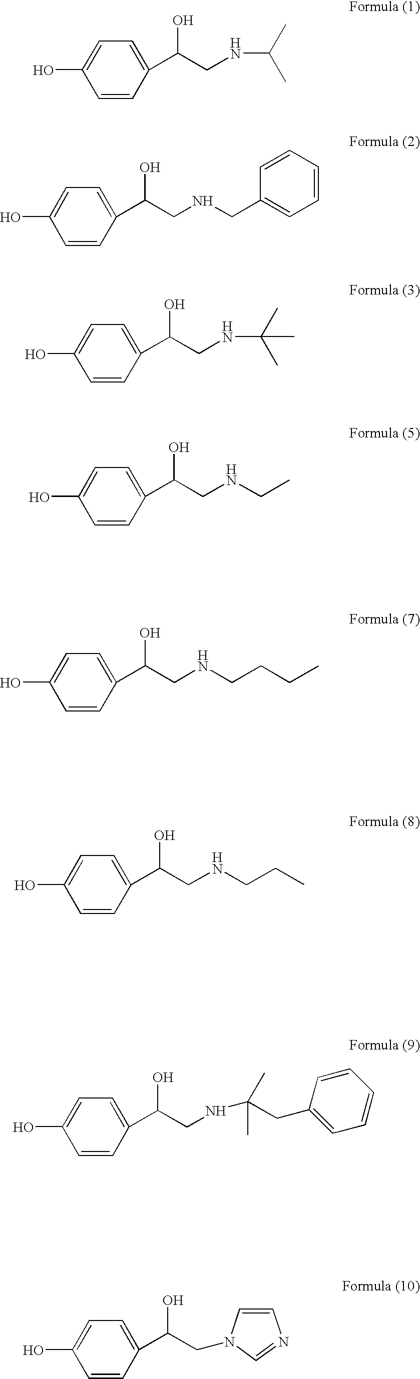

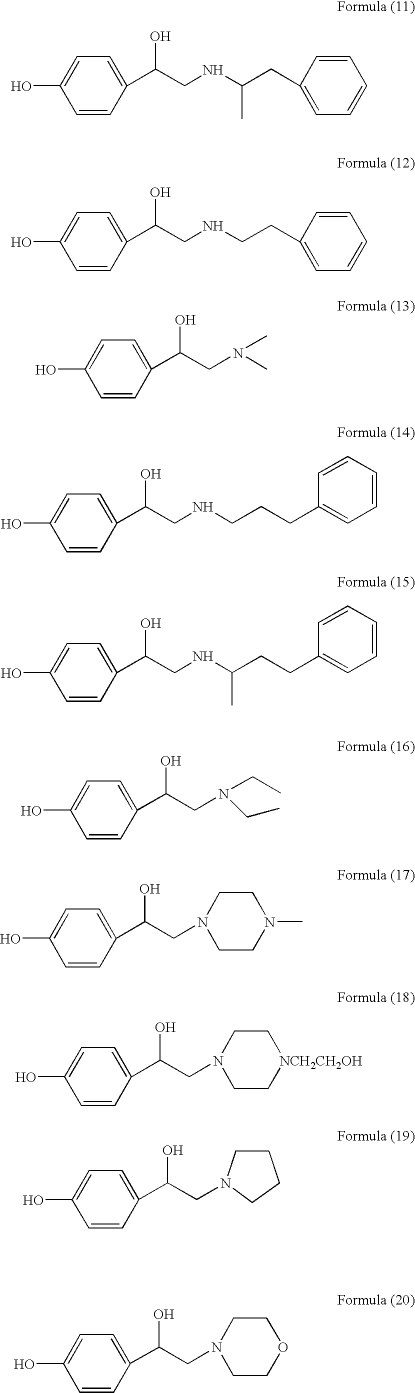

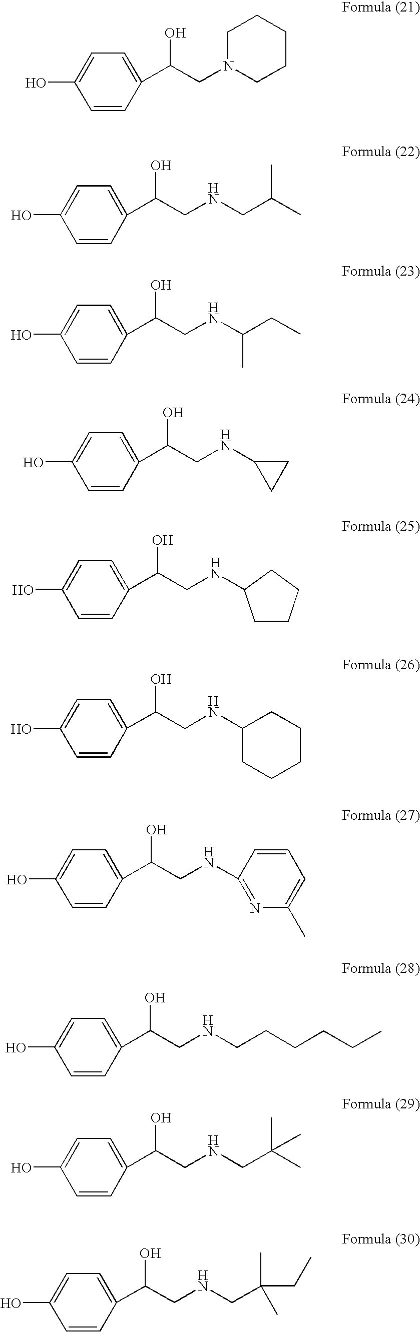

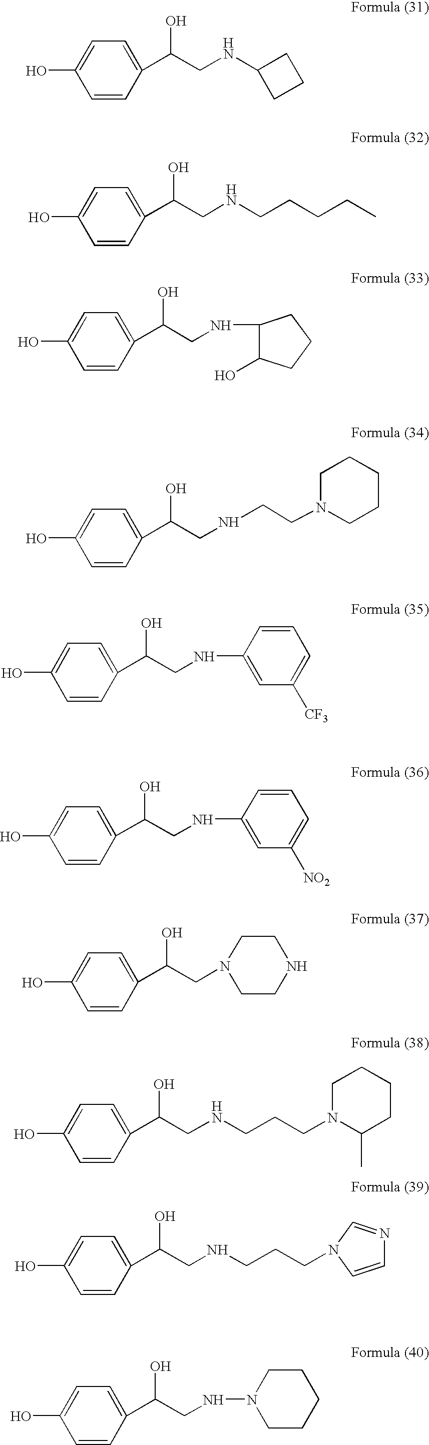

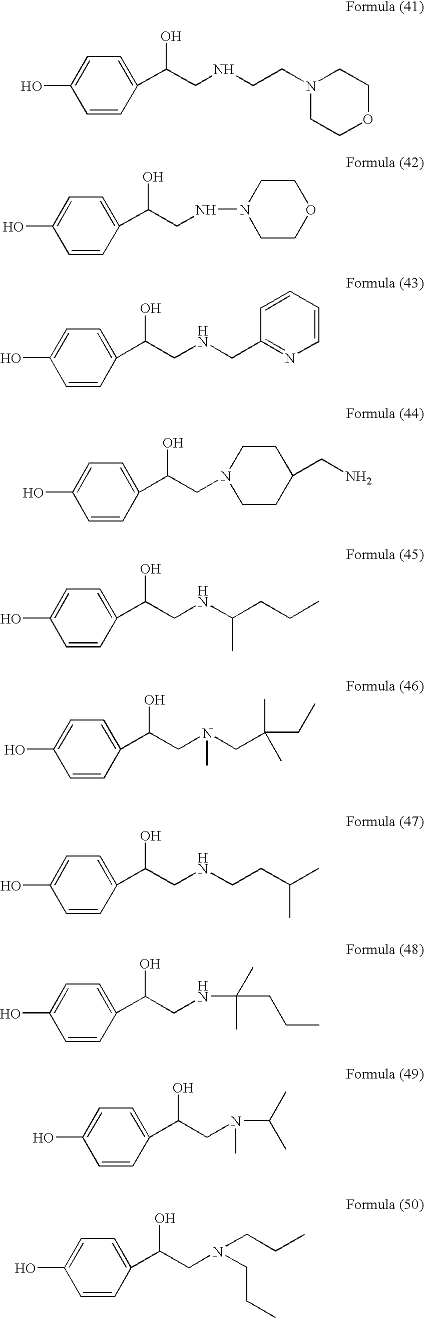

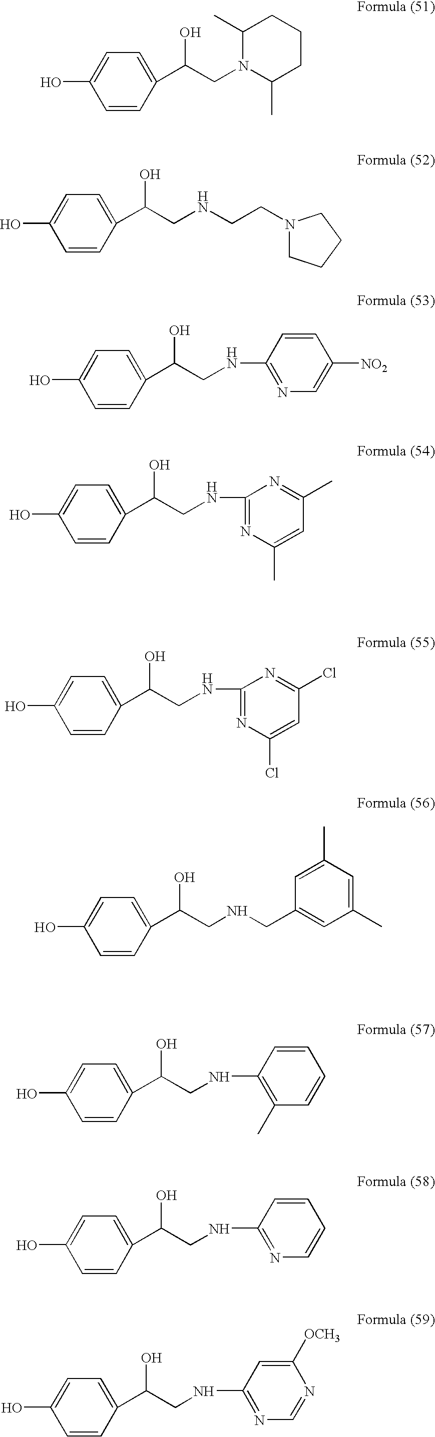

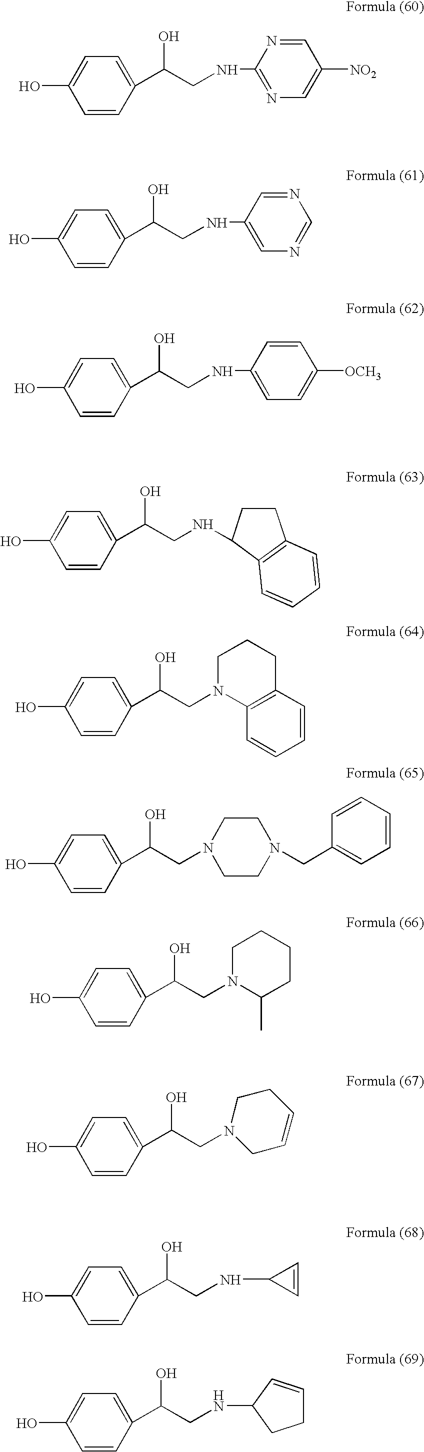

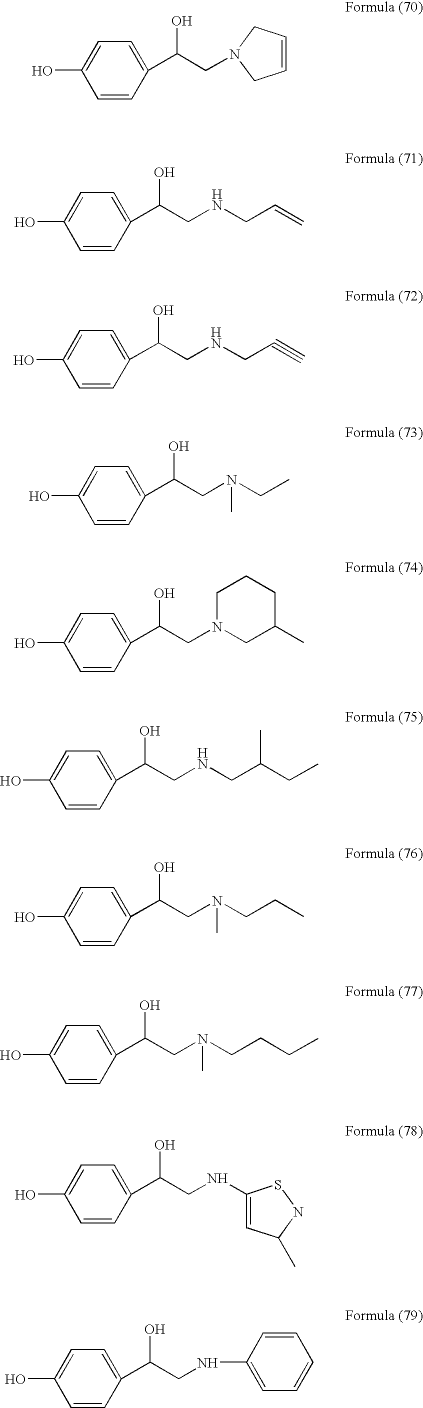

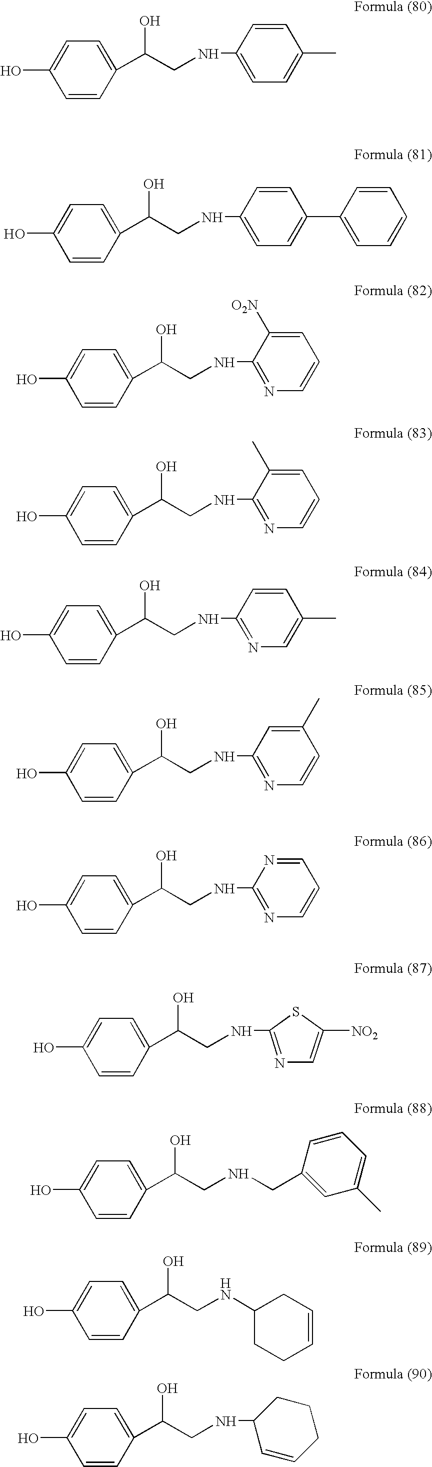

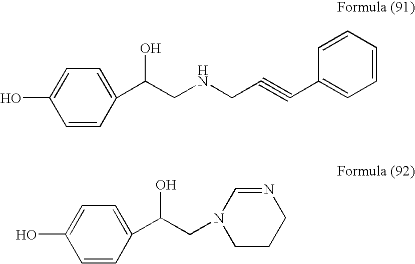

20. The pharmaceutical composition of claim 19, wherein the compound of Formula I is selected from the group consisting of Formulae: (1), (2), (3), (5), (7), (8), (9), (33), and (37).

21. The use of the pharmaceutical composition of claim 17 to stimulate body fat breakdown or weight loss in the mammal.

22. The use of claim 21, wherein the mammal is a human.

23. A method for synthesizing a compound of Formula VI, comprising the step of reacting a compound of Formula III with a base, to generate the compound of Formula VI, as graphically presented below: 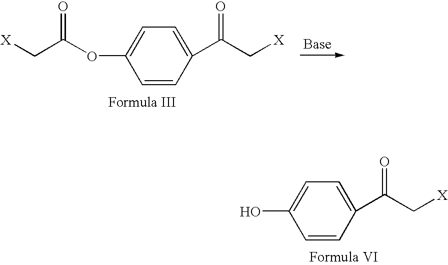
wherein X is a halo substituent selected from the group consisting of chloro, bromo, iodo, and fluoro.

24. The method for synthesizing the compound of Formula VI of claim 23, further comprising the step of first synthesizing the compound of Formula III by reacting a phenol of Formula II with a haloacetyl halide, in the presence of a Lewis acid catalyst, to produce the compound of Formula III, the reaction step is graphically presented below: 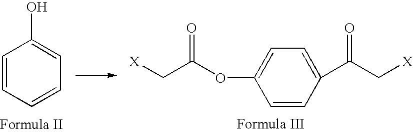
wherein X is as defined in claim 23.
